(19) United States
(12) Patent Application Publication
(10) Pub. No.: US 2026/0258121 A1
(43) Pub. Date: Sep. 3, 2026

(54) USES OF POMC-SPECIFIC ANTIBODY AND MARCHF6

(71) Applicant: KOREA UNIVERSITY RESEARCH AND BUSINESS FOUNDATION, Seoul (KR)

(72) Inventors: Cheol-Sang HWANG, Seoul (KR); Sang-Hyeon MUN, Seoul (KR)

(73) Assignee: KOREA UNIVERSITY RESEARCH AND BUSINESS FOUNDATION, Seoul (KR)

(21) Appl. No.: 19/489,958

(22) PCT Filed: Jun. 7, 2024
(86) PCT No.: PCT/KR2024/007779

§ 371 Date: Dec. 4, 2025

(30) Foreign Application Priority Data
Jun. 8, 2023 (KR) 10-2023-0073579
Oct. 6, 2023 (KR) 10-2023-0133189
Oct. 6, 2023 (KR) 10-2023-0133190

(51) Int. Cl. C07K 16/26; C07K 14/575; G01N 33/68

(52) U.S. Cl. CPC C07K 16/26; C07K 14/575; G01N 33/6893; G01N 2333/665

(57) ABSTRACT

The present disclosure relates to a POMC-specific antibody and use thereof, and use of Marchf6 for treating metabolic diseases. It has been found that the cytosolic accumulation of POMC, which is an appetite-regulating hormone precursor protein, induces ER stress and ferroptosis, and Marchf6 degrades POMC by directly binding thereto, causing the translocation of POMC into ER. Also, it has been confirmed that a produced POMC-specific antibody can specifically detect POMC accumulated in the cytosol of POMC neurons. Therefore, these can be used for the diagnosis and treatment of diseases associated with POMC accumulation or Marchf6 dysfunction. It has also been found that ER stress and ferroptosis caused by accumulation of POMC in the cytosol are regulated. In particular, it was confirmed in vivo that Marchf6 deficiency in POMC neurons causes metabolic disorders. Therefore, Marchf6 can be effectively used for the prevention or treatment of metabolic diseases, including obesity.

### TECHNICAL FIELD

The present disclosure relates to a POMC-specific antibody, particularly a POMC-specific antibody remaining in the cytosol, and use thereof, and use of Marchf6 for the treatment of metabolic diseases.

### BACKGROUND ART

Metabolic diseases are a general term for diseases caused by metabolic disorders in the body. Generally, the metabolic diseases are caused by an imbalance of carbohydrates, lipids, proteins, vitamins, electrolytes, and water, and examples thereof include obesity, diabetes, hyperlipidemia, arteriosclerosis, fatty liver, hypertension, etc. Metabolic diseases are also called lifestyle diseases because the imbalance of energy metabolism in vivo due to high-calorie, high-fat, and high-carbohydrate diets may cause obesity and induce insulin resistance and metabolic inflammation, which can lead to degenerative diseases such as lipid metabolism disorders and type 2 diabetes. Recently, in Korea, due to the westernization of the diet, the morbidity of chronic degenerative diseases such as hypertension, heart disease, arteriosclerosis, and diabetes is increasing due to changes in diet caused by increased intake of processed foods and animal-based foods and decreased intake of plant-based foods.

Obesity (typically defined as a body mass index of approximately >30 kg/m²) is a type of metabolic disorder caused by an imbalance between caloric intake and expenditure, and is often associated with various pathological conditions such as hyperinsulinemia, insulin resistance, diabetes, hypertension, and dyslipidemia (Mantzoros et al, J Clin Endocrinol Metab, 85:4000-2, 2000). As the incidence of obesity has rapidly increased over the past several decades, while interest in obesity has continued to grow, it is known that obesity occurs by excessive energy supply which causes an increase in the size and number of fat cells to be accumulated as in vivo fat. In addition, it is known that the obesity occurs due to various other causes, such as genetic factors, environmental factors due to a westernized diet, psychological factors, and energy metabolism abnormalities. If the obesity state persists for a long time, the obesity not only causes discomfort in physical activity, decreased work efficiency, and abnormal growth in physical development, but also causes various diseases, such as diabetes, hyperlipidemia, elevated blood cholesterol, kidney disease, heart disease, stroke, arteriosclerosis, fatty liver disease, coronary artery disease, and joint disease.

Many organs/tissues have been associated with the development of obesity and type 2 diabetes, and particularly, the hypothalamus was known to play a key role in energy homeostasis, including a control of energy intake, etc. (Stellar, Psychol Rev 61:5-22, 1954). The hypothalamus regulates the body weight by precisely balancing food intake, energy expenditures, and body fat tissue mass. The main hypothalamic areas involved in energy regulation (causing hypothalamic obesity when impaired) include the ventromedial hypothalamus, paraventricular nucleus, arcuate nucleus, and lateral hypothalamic areas. In addition, signals (including GLP-1, PYY, and/or pancreatic insulin/amelin) from adipose tissue mass (including leptin) within the body and from the gastrointestinal tract affect the hypothalamic center. Disorders that involve signaling interactions with the hypothalamus or damage to the hypothalamus may cause pathological hypothalamic obesity. Weight gain due to hypothalamic obesity occurs from destruction of normal homeostatic function of the hypothalamic center, together with loss of control of satiety and hunger, inability of energy balance regulation and/or reduction in energy expenditures, and/or frequent progression to hyperinsulinemia and diabetes. Obesity, which is caused by dysfunction in the hypothalamus, resulting in failure of intake control, causes excessive overeating that is difficult to tolerate, unlike the weight gain in normal obesity, and typically does not respond to diet and exercise. Such hypothalamic obesity may be caused by any damage or defect in the hypothalamus. The hypothalamic obesity may occur in patients with genetic syndromes, such as mutations in leptin or leptin receptors, cocaine and amphetamine-related transcript (CART), proopiomelanocortin (POMC), prohormone convertase, melanocortin-4 receptor (MC4R), singleminded 1 (a transcription factor essential for the formation of the supraoptic nucleus and PVN nuclei in the hypothalamus), or TrkB. In addition, the hypothalamic obesity may also be caused by Prader-Willi syndrome and Bardet-Biedl syndrome (BBS), which are caused by deletions of paternally imprinted genes on chromosome 15q11-q13.

### DISCLOSURE

### Technical Problem

An object of the present disclosure is to provide an antigenic peptide for producing a pro-opiomelanocortin (POMC)-specific antibody.

Another object of the present disclosure is to provide a composition for producing a POMC-specific antibody.

Yet another object of the present disclosure is to provide a method for producing a POMC-specific antibody.

Still another object of the present disclosure is to provide a POMC-specific antibody or antigen-binding fragment.

Still another object of the present disclosure is to provide a pharmaceutical composition for preventing or treating diseases associated with POMC accumulation.

Still another object of the present disclosure is to provide a pharmaceutical composition for preventing or treating diseases associated with Marchf6 dysfunction.

Still another object of the present disclosure is to provide a method for providing information for diagnosing metabolic diseases.

Still another object of the present disclosure is to provide a pharmaceutical composition for preventing or treating metabolic diseases.

Still another object of the present disclosure is to provide a composition for promoting POMC degradation.

Still another object of the present disclosure is to provide a composition for promoting translocation into ER.

Still another object of the present disclosure is to provide a composition for inhibiting ER stress or ferroptosis.

Still another object of the present disclosure is to provide a food composition for preventing or improving metabolic diseases.

Still another object of the present disclosure is to provide a method for screening an obesity therapeutic agent.

### Technical Solution

In order to achieve the objects, an aspect of the present disclosure provides an antigenic peptide for producing a pro-opiomelanocortin (POMC)-specific antibody.

Another aspect of the present disclosure provides a composition for producing a POMC-specific antibody.

Yet another aspect of the present disclosure provides a method for producing a POMC-specific antibody.

Still another aspect of the present disclosure provides a POMC-specific antibody or antigen-binding fragment.

Still another aspect of the present disclosure provides a pharmaceutical composition for preventing or treating diseases associated with POMC accumulation.

Still another aspect of the present disclosure provides a pharmaceutical composition for preventing or treating diseases associated with Marchf6 dysfunction.

Still another aspect of the present disclosure provides a method for providing information for diagnosing metabolic diseases.

Still another aspect of the present disclosure provides a pharmaceutical composition for preventing or treating metabolic diseases.

Still another aspect of the present disclosure provides a composition for promoting POMC degradation.

Still another aspect of the present disclosure provides a composition for promoting translocation into ER.

Still another aspect of the present disclosure provides a composition for inhibiting ER stress or ferroptosis.

Still another aspect of the present disclosure provides a food composition for preventing or improving metabolic diseases.

Still another aspect of the present disclosure provides a method for screening an obesity therapeutic agent.

### Advantageous Effects

According to the present disclosure, it has been found that Marchf6 directly recognizes POMC, which is an appetite-regulating hormone precursor protein, to degrade POMC and regulate the translocation of POMC into ER, thereby regulating ER stress and ferroptosis caused by accumulation of POMC in the cytosol, and specific mechanisms thereof have been identified. In particular, it was confirmed in vivo that Marchf6 deficiency in POMC neurons causes metabolic disorders, such as obesity, bulimia, increased appetite and decreased energy metabolism.

Therefore, Marchf6 can be effectively used for the prevention or treatment of metabolic diseases, including obesity.

In addition, it has been found that the cytosolic accumulation of POMC, which is an appetite-regulating hormone precursor protein, induces ER stress and ferroptosis, and Marchf6 degrades POMC by directly binding thereto, causing the translocation of POMC into ER. Also, it has been confirmed that a POMC-specific antibody remaining in the cytosol is produced to specifically detect POMC accumulated actually in the cytosol of POMC neurons. Therefore, these can be effectively used for the diagnosis and treatment of diseases associated with POMC accumulation or Marchf6 dysfunction.

### DESCRIPTION OF DRAWINGS

FIG. 1 is a diagram illustrating screening of a Marchf6 binding protein:
A: Screening experiment process;
B: Putative domain-specific binding protein of Marchf6; and
C: Split-Ub assay using full-length Marchf6 as bait and POMC, Bnip3, Gps1, SNAPIN, Yif1a, Hspa5, Hax1, Fh12, and Mcfd2 as preys.

FIG. 2 is a diagram illustrating investigation of interacting sites between Marchf6 and POMC:
A: Schematic diagram and sequence of Marchf6;
B: Y2H assay;
C: Split-Ub assay;
D and E: Reciprocal co-immunoprecipitation of Marchf63f or Marchf63f P460A with POMCₕₐ (*: nonspecific band);
F: CHX-chase assay of POMC and SM;
G: Immunoblotting of POMCₕₐ and Marchf63f; and
H: In vivo ubiquitination assay of POMC_{myc} under MG132 (10 μM) treatment for 8 hours.

FIG. 3 is a diagram illustrating analysis of a POMC degradation mechanism of Marchf6:
A: Immunoblotting of endogenous Marchf6;
B: CHX-chase assay of POMCₕₐ;
C: CHX-chase assay of endogenous POMC;
D: In vivo ubiquitination assay of POMC_{myc} under MG132 (10 M) treatment for 8 hours;
E: CHX-chase assay of POMCₕₐ after treatment with Bag6, Derl1, or VCP siRNA; and
F: Relative mRNA levels of POMCₕₐ after treatment with Bag6, Derl1, or VCP siRNA.

FIG. 4 is a diagram illustrating confirming a POMC recognition site of Marchf6:
A: Y2H assay (left) and configuration of POMC (right);
SP: Signal peptide;
N-POMC: Nt-POMC domain;
ACTH: Adrenocorticotropic hormone;
β-LPH: β-lipotropin;
B: Split-Ub assay using Marchf6 as bait and POMC fragment as prey;
C: GST pulldown assay;
D: Epitope schematic diagram for producing antibody (anti-POMC^{SP}) against POMC containing SP;
E: Immunoblotting of anti-POMC^{SP} with GST-POMC¹⁻⁷⁶, GST-POMC²⁷⁻⁷⁶, and GST-POMC¹⁻²⁶.
F: Schematic diagram of IP and IB experiments after digitonin/Triton X-100-based cytosol-organelle fractionation; and
G: Results of IP using anti-POMC^{SP} and IB using anti-HA after digitonin/Triton X-100-based cytosol-organelle fractionation.

FIG. 5 is a diagram illustrating ER stress and ferroptosis inhibition effects of Marchf6 by POMC:
A: Lipid ROS levels with incremental expression of POMCₕₐ;
B: Relative NADP(H) levels with incremental expression of POMCₕₐ;
C: Relative cell viability with incremental expression of POMCₕₐ;
D: Relative LDH release with incremental expression of POMCₕₐ;
E: Immunoblotting of Gpx4, CHOP, Nox2, and Nox4 with incremental expression of POMCₕₐ;
F: Immunoblotting of 4-HNE, Gpx4, CHOP, Nox2, and Nox4 with or without 24-h treatment with Fer-1 (5 μM);
G: Lipid ROS level;
H: Relative cell viability;
I: Immunoblotting of Gpx4, CHOP, Nox2, and Nox4; and
J: Relative cell viability in the presence of Fer-1 (5 μM), Z-VAD (20 μM), or Nec-1 (40 M).

FIG. 6 is a diagram illustrating analysis of a mechanism in which cytosolic POMC induces ER stress and ferroptosis:
A: Lipid ROS level;
B to D: NADP(H) level, cell viability, and LDH release;
E: Immunoblotting of Gpx4, CHOP, Nox2, Nox4, Atf4, eIF2α-p (phosphorylated eIF2α), eIF2α, PERK-p (phosphorylated PERK), PERK, Ire1-p (phosphorylated Ire1), Ire1, and Atf6;
F: Relative Gpx4 mRNA level;
G: CHX-chase assay of Gpx4;
H: Immunoblotting of Gpx4 in cells treated with MG132 (20 μM), 3-MA (5 mM), BafA1 (100 nM), or CQ (50 μM) for 8 hours;
I: Schematic diagram of predicted cytosolic POMC-induced Gpx4 degradation via CMA;
J: Immunoblotting of Hsp90, Hsc70, and Lamp2a; and
K: CHX-chase assay of Gpx4 after siRNA treatment against Lamp2a.

FIG. 7 is a diagram illustrating analysis of inhibition of Hspa5-Gpx4 interaction by cytosolic POMC:
A: CHX-chase assay of Gpx4;
B: Gpx4 mRNA level;
C: Immunoblotting of Hspa5, Gpx4, tubulin (cytosolic marker), calreticulin (ER lumen marker), Sec61α (ER membrane marker), and Na⁺/K⁺ ATPase (plasma membrane marker) after digitonin/Triton X-100-based cytosolic/organelle fractionation;
D: Reciprocal co-immunoprecipitation assay of POMCₕₐ^{Δ1-26} and Hspa5_{myc};
E: Immunoprecipitation of Gpx4 using Hspa5_{myc};
F: Immunoprecipitation of Gpx4 using anti-Hspa5 antibody with or without Fer-1 (5 M) treatment for 24 hours;
G: Co-immunoprecipitation with anti-POMC^{SP} and immunoblotting using anti-HA after digitonin/Triton X-100-based cytosol/organelle fractionation with or without Fer-1 (5 μM) treatment; and
H: Co-immunoprecipitation with anti-POMC^{SP} and immunoblotting using anti-HA after digitonin/Triton X-100-based cytosol/organelle fractionation with or without erastin (10 μM) treatment for 24 hours.

FIG. 8 is a diagram illustrating an inhibitory effect of POMC-induced Gpx4 degradation by cytosolic Hspa5:
A: Immunoblotting of ferroptosis effectors Acsl4, Alox5, TfR1, POR, Nox1, Lpcat3, Slc40a1, Fspl, HO1, and Nrf2;
B: Lipid ROS level;
C: Cell viability;
D: Immunoblotting of Gpx4, CHOP, Nox2, and Nox4; and
E: CHX-chase assay of Gpx4.

FIG. 9 is a diagram illustrating hyperphagia, decreased metabolic rate, and weight gain in Marchf6^{POMC} mice, which are POMC neuron-specific Marchf6-deficient mice.
A: Growth curve of male mice;
B: Growth curve of female mice;
C: Daily food intake of 20-week-old male mice;
D: Daily food intake of 20-week-old female mice;
E: Energy expenditure of 20-week-old male mice;
F: Energy expenditure of 20-week-old female mice; and
G to I: Quantitative data for immunohistochemically stained 4-HNE (G), Gpx4 (H), and Hspa5 (I) in mouse POMC neurons.

FIG. 10 is a diagram illustrating analysis of weight gain of Marchf6^{POMC} mice:
A: Body weight of 20-week-old male mice;
B: Body weight of 20-week-old female mice;
C: Fat mass of 20-week-old male mice;
D: Fat mass of 20-week-old female mice;
E: Lean mass of 20-week-old male mice;
F: Lean mass of 20-week-old female mice.

FIG. 11 is a diagram illustrating IHC assay of 4-HNE, Hspa5, and Gpx4 in POMC neurons of Marchf6^{POMC} mice and littermate Marchf6^{f1/f1} mice.

FIG. 12 is a schematic diagram illustrating a mechanism in which Marchf6 regulates ER stress, ferroptosis, and metabolic homeostasis in POMC neurons.

### BEST MODE

Hereinafter, embodiments of the present disclosure will be described in detail with reference to the accompanying drawings. However, the following embodiments are presented as examples for the present disclosure, and when it is determined that the detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

In addition, terminologies used herein are terminologies used to properly express preferred embodiments of the present disclosure, which may vary according to a user, an operator's intention, or customs in the art to which the present disclosure pertains. Therefore, these terminologies used herein will be defined based on the contents throughout the specification. Throughout this specification, unless explicitly described to the contrary, when a certain part “comprises” a certain component, it will be meant to further comprise other components rather than excluding other components.

All technical terms used in the present disclosure, unless otherwise defined, are used in the meaning as commonly understood by those skilled in the related art of the present disclosure. In addition, although preferred methods and samples are described herein, similar or equivalent methods and samples thereto are also included in the scope of the present disclosure. The contents of all publications described herein as references are incorporated in the present disclosure.

Throughout the present specification, general one-letter or three-letter codes for naturally occurring amino acids are used, and generally allowed three-letter codes for other amino acids, such as α-aminoisobutyric acid (Aib) and N-methylglycine (Sar) are also used. The amino acids mentioned herein as abbreviations are also described as follows according to the IUPAC-IUB nomenclature.

Alanine: A; Arginine: R; Asparagine: N; Aspartic acid: D; Cysteine: C; Glutamic acid: E; Glutamine: Q; Glycine: G; Histidine: H; Isoleucine: I; Leucine: L; Lysine: K; Methionine: M; Phenylalanine: F; Proline: P; Serine: S; Threonine: T; Tryptophan: W; Tyrosine: Y; and Valine: V.

In one aspect, the present disclosure relates to an antigenic peptide for producing a pro-opiomelanocortin (POMC)-specific antibody, including an amino acid sequence represented by SEQ ID NO: 1.

In an embodiment, the antigenic peptide may be an isolated peptide, and may be an epitope or epitope segment.

In an embodiment, the antigenic peptide may be an antigenic peptide for producing a POMC-specific antibody including a signal peptide (SP) sequence.

In an embodiment, the antigenic peptide may further include a carrier protein, and the carrier protein may be Keyhole-Limpet Hemocyanin (KLH), bovine serum albumin (BSA), or ovalbumin (OVA).

In an embodiment, the carrier protein may be linked to the antigenic peptide via a linker, and the linker may be a chemical linker.

As used herein, the “peptide” is a polymer of amino acids, and usually, a form in which a small number of amino acids are linked, called a peptide, and a form in which many amino acids are linked, called a protein. The linkage between amino acids in a peptide or protein structure consists of amide bonds or peptide bonds. The peptide bond refers to a bond in which water (H₂O) is removed between a carboxyl group (—COOH) and an amino group (—NH₂) to have a —CO—NH— form. The peptide of the present disclosure may be prepared according to chemical synthesis methods known in the art, especially solid-phase synthesis techniques (Merrifield, J. Amer. Chem. Soc. 85:2149-54(1963); Stewart, et al., Solid Phase Peptide Synthesis, 2nd. ed., Pierce Chem. Co.: Rockford, 111 (1984)), and also produced by genetic engineering techniques.

In the present disclosure, even if the peptide is described as a ‘peptide consisting of a specific sequence number’, if the peptide has the same or corresponding activity as or to a peptide consisting of the amino acid sequence represented by the corresponding sequence number, it is obvious that without excluding the addition of meaningless sequences before or after the amino acid sequence represented by the corresponding sequence number, mutations that may occur naturally, or silent mutations thereof, such sequence additions or mutations fall within the scope of the present disclosure.

In the present disclosure, in the modification of the peptide sequence, some amino acids may be modified through any one of substitution, addition, deletion, and modification, or a combination of these methods. These modifications include modifications using L-type or D-type amino acids, and/or non-natural amino acids; and/or modifications by modifying a native sequence, such as modification of side-chain functional groups, intramolecular covalent bonding, such as inter-side chain ring formation, methylation, acylation, ubiquitination, phosphorylation, aminohexation, biotinylation, etc. As the substituted or added amino acids, not only 20 amino acids conventionally observed in human proteins, but also atypical or non-naturally occurring amino acids may be used. Commercial sources of the atypical amino acids include Sigma-Aldrich, ChemPep, and Genzyme pharmaceuticals. The sequences for the peptide including these amino acids and the typical peptide can be synthesized and purchased through commercialized peptide synthesis companies, such as American Peptide Company or Bachem in USA, or Anygen in Korea.

In addition, the range of the antigenic peptide of the present disclosure includes functional equivalents of the peptide including the amino acid sequence represented by SEQ ID NO: 1, more preferably functional equivalents of the peptide including the amino acid sequence represented by SEQ ID NO: 1, and salts thereof.

The functional equivalents refer to peptides which have sequence homology (that is, identity) of at least 75% or more, preferably 90%, more preferably 95% or more with the peptide represented by SEQ ID NO: 1, for example, sequence homology of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%, as a result of addition, substitution or deletion of amino acids, and exhibit substantially the same physiological activity as the peptide represented by SEQ ID NO: 1. In the present specification, the sequence homology and homogeneity are defined as a percentage of amino acid residues of a candidate sequence to the amino acid sequence represented by SEQ ID NO: 1 after aligning the amino acid sequence represented by SEQ ID NO: 1 and the candidate sequence and introducing gaps. If necessary, conservative substitution is not considered as a part of sequence homogeneity in order to obtain the maximum percentage sequence homogeneity. An N-terminal, a C-terminal or internal extension, deletion or insertion of the amino acid sequence represented by SEQ ID NO: 1 is not construed as a sequence affecting sequence homogeneity or homology.

In addition, the sequence homogeneity may be determined by general standard methods used for comparing similar portions of amino acid sequences of two polypeptides. A computer program such as BLAST or FASTA aligns the two polypeptides so as to optimally match respective amino acids (according to a full-length sequence of one or two sequences, or a predicted portion of one or two sequences). The program provides a default opening penalty and a default gap penalty and provides a scoring matrix such as PAM250 (standard scoring matrix) which may be used in association with the computer program. For example, the percentage homogeneity may be calculated as follows. The total number of identical matches is multiplied by 100 and then divided by a sum of the length of a longer sequence in a corresponding matched span and the number of gaps introduced into the longer sequence to align the two sequences.

The scope of functional equivalents of the present disclosure includes derivatives and mimetics/peptidomimetics. The “derivative” refers to a general term for similar peptides that have some modified chemical structures while maintaining the basic skeleton of the peptide represented by SEQ NO: 1, and preferably, peptides in which one or more amino acids may be substituted with other amino acids, one or more amino acids may be added, one or more amino acids may be deleted, or a compound (e.g., polyethylene glycol, etc.) that increases the half-life of the peptide may be fused. As used herein, the derivatives may maintain, increase, or decrease the stability, storability, volatility, solubility, or the like of the peptide according to the present disclosure.

In one aspect, the present disclosure relates to a composition for producing a POMC-specific antibody, including the antigenic peptide of the present disclosure.

In an embodiment, the composition may be a reagent composition for producing the POMC-specific antibody.

In one aspect, the present disclosure relates to a kit for producing a POMC-specific antibody, including the antigenic peptide of the present disclosure.

The composition and the kit for producing the antibody according to the present disclosure may further include known ingredients for maintaining and preserving the antigenic peptide.

In an embodiment, the composition and the kit for producing the antibody may further include a known agent for inducing and promoting immune responses in a host.

In one aspect, the present disclosure relates to a method for producing a POMC-specific antibody, including inducing an immune response by inoculating a host other than a human multiple times with an antigenic peptide of the present disclosure or a composition including the antigenic peptide; and obtaining serum from the blood of the host.

In an embodiment, the host may be a mammal other than a human, and the mammal other than the human may be mouse, rabbit, rat, guinea pig, horse, dog, sheep, goat, cat, chicken, duck, monkey or primate, and more preferably rabbit, but is not limited thereto.

In an embodiment, the method may further include purifying the POMC-specific antibody from the serum.

In one aspect, the present disclosure relates to a POMC-specific antibody or antigen-binding fragment that specifically binds to POMC produced by the method of the present disclosure.

In an embodiment, the POMC-specific antibody or antigen-binding fragment of the present disclosure may specifically bind to POMC including an SP sequence.

In an embodiment, the POMC-specific antibody of the present disclosure may be a polyclonal antibody.

In an embodiment, the POMC-specific antibody or antigen-binding fragment of the present disclosure may bind to an epitope or epitope segment including the amino acid sequence represented by SEQ ID NO: 1.

In an embodiment, the POMC-specific antibody or antigen-binding fragment of the present disclosure may specifically bind to POMC present in the cytosol of POMC neurons.

In an embodiment, the POMC-specific antibody or antigen-binding fragment of the present disclosure may be used as a POMC neuron targeting composition in which POMC is accumulated in the cytosol.

In an embodiment, the POMC-specific antibody or antigen-binding fragment may also include a tag, a labeled residue, or an additional amino acid sequence designed for the specific purpose of increasing the half-life or stability of the protein.

In an embodiment, the tag may be a His tag, a Myc (c-myc) tag, a FLAG tag, an HA tag, or a T7 tag.

In an embodiment, the POMC-specific antibody or antigen-binding fragment of the present disclosure may further include a detectable label. The label may be a fluorescent label, a chemiluminescent label, an enzymatic label, and a radionuclide label. The fluorescent label may be green fluorescent protein (GFP), enhanced green fluorescent protein (EGFP), yellow fluorescent protein (YFP), red fluorescent protein (RFP), orange fluorescent protein (OFP), cyan fluorescent protein (CFP), blue fluorescent protein (BFP), far-red fluorescent protein, or a tetracysteine motif.

In an embodiment, the POMC-specific antibody or antigen-binding fragment may be further conjugated to RNA, DNA, an antibody, an effector, a drug, a prodrug, a toxin, a peptide or a delivery molecule (see Shoari et al., Pharmaceutics 13:1391, pp. 1-32 (2021)).

In an embodiment, the drug may also be genes, plasmid DNA, antisense oligonucleotide, siRNA, peptides, ribozyme, viral particles, immunomodulators, proteins, contrast agents, or the like.

The conjugate of the POMC-specific antibody or antigen-binding fragment of the present disclosure and the drug may be prepared as a pharmaceutical composition in the form of an oral formulation or a parenteral formulation depending on a route of administration by a conventional method known in the art, including the pharmaceutically acceptable carrier. As used herein, the “pharmaceutically acceptable carrier” may refer to a carrier or a diluent which does not inhibit biological activity and properties of a compound to be administered without stimulating organisms. In the composition formulated with a liquid solution, the pharmaceutically acceptable carrier is suitable for sterilization and living bodies, and may include saline, sterilized water, Ringer's solution, buffered saline, albumin injection solution, dextrose solution, maltodextrin solution, glycerol, ethanol, and a mixture of at least one of these ingredients. If necessary, other general additives, such as antioxidants, buffers, bacteriostatic agents, and the like, may be added.

As used herein, the term “antibody” refers to a functional ingredient of serum and is often referred to as a collection of molecules (antibodies or immunoglobulin) or a single molecule (antibody molecule or immunoglobulin molecule). The antibody molecule may bind to or react with a specific antigenic determinant (antigen or antigenic epitope) to sequentially induce an immunological effector mechanism.

An individual antibody molecule is generally considered to be monospecific, and a composition of antibody molecules may be monoclonal (i.e., composed of identical antibody molecules) or polyclonal (i.e., composed of different antibody molecules that react with the same or different epitopes on the same antigen or on separate antigens). Each antibody molecule has a unique structure capable of binding specifically to a corresponding antigen thereto, and all natural antibody molecules have the same overall basic structure of two identical light chains and two identical heavy chains. The antibody is also collectively known as immunoglobulin. As used herein, the term of the antibody or antibodies is used in the broadest sense and includes not only intact antibodies, chimeric antibodies, humanized antibodies, complete human and single-chain antibodies, but also binding fragments of the antibody, such as Fab, Fv fragment or scFv fragment, and multimeric forms, such as dimeric IgA molecule or pentavalent IgM.

As used herein, the term “specifically binding” or “specifically recognizing” has the same meaning as commonly known to those skilled in the art, and means that an antigen and an antibody specifically interact with each other to have an immunological response.

As used herein, the “epitope” is generally used to describe a portion of a larger molecule or a part of a larger molecule (e.g., antigen or antigenic site) that has antigenic or immunogenic activity in an animal, preferably a mammal, and most preferably a human. The epitope having immunogenic activity is a portion of a larger molecule that causes an antibody response in an animal. The epitope having the antigenic activity is a portion of a larger molecule which immunospecifically binds to an antibody determined by any method well-known in the art, for example, by an immunoassay described herein. The antigenic epitope is not necessarily immunogenic. The antigen is a substance, which immunospecifically binds to an antibody or antibody fragment, such as toxin, virus, bacteria, protein, or DNA. The antigen or antigenic site often has more than one epitope, unless very small, and may often stimulate an immune response. Antibodies that bind to different epitopes on the same antigen may have various effects on the activity of antigens to which these antibodies bind, depending on a location of the epitope. An antibody that binds to the epitope at the active site of the antigen completely blocks the function of the antigen, whereas another antibody that binds to a different epitope may have little or no effect on the activity of the antigen.

As used herein, the term “polyclonal antibody” refers to a composition of different (various) antibody molecules capable of binding to or reacting with several different specific antigenic determinants/epitopes on the same or different antigens, and each antibody in the composition may react with a specific epitope. In general, the variability of the polyclonal antibody is present in so-called variable regions of the polyclonal antibody, particularly CDR1, CDR2 and CDR3 regions. In the present disclosure, the polyclonal antibody may be produced in one pot or may be a mixture of different polyclonal antibodies. A mixture of monoclonal antibodies is produced in individual batches and is not necessarily derived from the same organism or cell line. This would result in differences in post-translational modification, and therefore, the mixture itself is not considered a polyclonal antibody. However, if the mixture of monoclonal antibodies provides the same antigen/epitope coverage as the polyclonal antibody of the present disclosure, it would be considered equivalent to the polyclonal antibody.

In one aspect, the present disclosure relates to a pharmaceutical composition for preventing or treating diseases associated with POMC accumulation, including a POMC-specific antibody or antigen-binding fragment of the present disclosure as an active ingredient.

In an embodiment, the diseases associated with POMC accumulation may be diseases in which POMC is accumulated in the cytosol of POMC neurons, and may be metabolic diseases.

In an embodiment, the metabolic diseases may be any one selected from the group consisting of obesity, bulimia, diabetes, arteriosclerosis, hypertension, hyperlipidemia, fatty liver, metabolic liver disease, and cardiovascular disease, preferably obesity, and more preferably obesity caused by a hypothalamic disorder.

In an embodiment, the obesity caused by the hypothalamic disorder may be obesity caused by ER stress or ferroptosis caused by a POMC protein accumulated in the cytosol of POMC neurons in the hypothalamus, and the obesity may have symptoms of bulimia, increased appetite, and decreased energy metabolism.

In one aspect, the present disclosure relates to a pharmaceutical composition for preventing or treating diseases associated with membrane associated ring-CH-type finger 6 (Marchf6) dysfunction, including a POMC-specific antibody or antigen-binding fragment of the present disclosure as an active ingredient.

In an embodiment, the Marchf6 may function to degrade SP-containing POMC in the cytosol of POMC neurons to inhibit lipid peroxidation and cause translocation of POMC into ER.

In an embodiment, the diseases may be diseases in which the function of Marchf6, which degrades the SP sequence of cytosolic POMC, is reduced, and may be metabolic diseases, most preferably obesity.

As used herein, the term “prevention” means all actions of inhibiting or delaying the occurrence, spread, and recurrence of the corresponding disease by administering the pharmaceutical composition according to the present disclosure, and “treatment” means all actions of improving or beneficially changing the symptoms of the disease by administering the composition of the present disclosure. Those skilled in the art to which the present disclosure pertains will be able to determine the degree of improvement, enhancement and treatment by knowing the exact criteria of a disease for which the composition of the present disclosure is effective by referring to data presented by the Korean Medical Association, etc.

As used herein, the term “therapeutically effective amount” used in combination with the active ingredient means an amount effective to prevent or treat a target disease, and the therapeutically effective amount of the composition of the present disclosure may vary depending on several factors, such as a method of administration, a target site, the condition of a patient, etc. Accordingly, when used in the human body, a dose should be determined as an appropriate amount in consideration of both safety and efficiency. It is also possible to estimate an amount to be used in humans from the effective amount determined through animal experiments. These matters to be considered when determining the effective amount are described in, for example, Hardman and Limbird, eds., Goodman and Gilman's The Pharmacological Basis of Therapeutics, 10th ed. (2001), Pergamon Press; and E. W. Martin ed., Remington's Pharmaceutical Sciences, 18th ed. (1990), Mack Publishing Co.

The pharmaceutical composition of the present disclosure is administered in a pharmaceutically effective amount. As used herein, the term “pharmaceutically effective amount” refers to an amount enough to treat the disease at a reasonable benefit/risk ratio applicable to medical treatment and not to cause side effects. The effective amount level may be determined according to factors including the health condition of a patient, a type of disease, cause and severity of the disease, activity of a drug, sensitivity to the drug, method of administration, time of administration, route of administration and excretion rate, treatment period, drugs used in combination or concurrently, and other factors well-known in the medical field. The composition of the present disclosure may be administered as an individual therapeutic agent or in combination with other therapeutic agents, and may be administered sequentially or simultaneously with conventional therapeutic agents, and may be administered as a single dose or in multiple doses.

It is important to administer an amount capable of obtaining a maximum effect with a minimal amount without side effects by considering all the factors, which may be easily determined by those skilled in the art.

The pharmaceutical composition of the present disclosure may include carriers, diluents, excipients, or a combination of two or more thereof, which are commonly used in biological agents. As used herein, the term “pharmaceutically acceptable” means that the composition exhibits non-toxic properties to cells or humans exposed to the composition. The carrier is not particularly limited as long as the carrier is suitable for in vivo delivery of the composition, and may be used by combining, for example, compounds described in Merck Index, 13th ed., Merck & Co. Inc., saline, sterile water, a Ringer's solution, buffered saline, a dextrose solution, a maltodextrin solution, glycerol, ethanol, and one or more of these components, and if necessary, other conventional additives such as an antioxidant, a buffer, and a bacteriostat may be added. In addition, the pharmaceutical composition may be formulated in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be formulated preferably according to each disease or ingredient using a suitable method in the art or a method disclosed in Remington's Pharmaceutical Science (Mack Publishing Company, Easton PA, 18th, 1990).

In an embodiment, the pharmaceutical composition may be one or more formulations selected from the group consisting of oral formulations, external preparations, suppositories, sterile injections and sprays, and more preferably oral or injectable formulations.

As used herein, the term “administration” means providing a predetermined substance to a subject or patient by any suitable method, and the pharmaceutical composition may be administered parenterally (e.g., applied as an injectable formulation intravenously, subcutaneously, intraperitoneally or topically) or orally according to a desired method. The dose range may vary depending on the body weight, age, sex, and health condition of a patient, a diet, an administration time, an administration method, an excretion rate, the severity of a disease, etc. Liquid formulations for oral administration of the composition of the present disclosure correspond to suspensions, internal solutions, emulsions, syrups, etc., and may include various excipients, such as wetting agents, sweeteners, fragrances, preservatives, and the like, in addition to water and liquid paraffin, which are commonly used simple diluents. Formulations for parenteral administration include sterilized aqueous solutions, non-aqueous solvents, suspensions, emulsions, lyophilized agents, suppositories, and the like. The pharmaceutical composition of the present disclosure may also be administered by any device capable of translocating an active substance to a target cell. Preferred administration methods and formulations are intravenous injections, subcutaneous injections, intradermal injections, intramuscular injections, drop injections, etc. The injections may be prepared by using aqueous solvents such as a physiological saline solution and a Ringer's solution, and non-aqueous solvents such as vegetable oils, higher fatty acid esters (e.g., ethyl oleate), and alcohols (e.g., ethanol, benzyl alcohol, propylene glycol, or glycerin). The injections may include pharmaceutical carriers, such as a stabilizer (e.g., ascorbic acid, sodium hydrogen sulfite, sodium pyrosulfite, BHA, tocopherol, EDTA, etc.) for the prevention of degeneration, an emulsifier, a buffer for pH control, and a preservative (e.g., phenyl mercury nitrate, thimerosal, benzalkonium chloride, phenol, cresol, benzyl alcohol, etc.) to inhibit microbial growth.

As used herein, the term “subject” refers to all animals including monkey, cow, horse, sheep, pig, chicken, turkey, quail, cat, dog, mice, rats, rabbit or guinea pig including humans who have developed or may develop the corresponding disease, and the pharmaceutical composition of the present disclosure may be administered to a subject to effectively prevent or treat the diseases.

The pharmaceutical composition of the present disclosure may be administered in combination with existing therapeutic agents.

The pharmaceutical composition of the present disclosure may further include pharmaceutically acceptable additives. At this time, the pharmaceutically acceptable additives may be used with starch, gelatinized starch, microcrystalline cellulose, lactose, povidone, colloidal silicon dioxide, calcium hydrogen phosphate, lactose, mannitol, syrup, arabic gum, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, white sugar, dextrose, sorbitol, talc and the like. The pharmaceutically acceptable additive according to the present disclosure is preferably included in an amount of 0.1 part by weight to 90 parts by weight based on the composition, but is not limited thereto.

The pharmaceutical composition of the present disclosure may be used as a single therapy, but may also be used in combination with other conventional biological therapy or chemotherapy, and may treat more effectively the corresponding disease in the case of such a combined therapy.

In one aspect, the present disclosure relates to a composition for diagnosing diseases associated with POMC accumulation, including a POMC-specific antibody or antigen-binding fragment of the present disclosure as an active ingredient.

In an embodiment, the composition for diagnosing an antibody or immunologically active fragment thereof of the present disclosure may be used to detect the accumulation of POMC or measure the amount (level) thereof in a biological sample isolated from a subject.

As used herein, the term “detection” or “measurement” refers to the presence or absence of a detected or measured object or to quantifying its concentration.

In one aspect, the present disclosure relates to a composition for diagnosing diseases associated with Marchf6 dysfunction, including a POMC-specific antibody or antigen-binding fragment of the present disclosure as an active ingredient.

In one aspect, the present disclosure relates to a method for providing information for diagnosing metabolic diseases, including detecting POMC accumulated in the cytosol of POMC neurons in a biological sample isolated from a subject using a POMC-specific antibody or antigen-binding fragment of the present disclosure.

In an embodiment, the metabolic diseases may be any one selected from the group consisting of obesity, bulimia, diabetes, arteriosclerosis, hypertension, hyperlipidemia, fatty liver, metabolic liver disease, and cardiovascular disease, and more preferably obesity.

In one aspect, the present disclosure relates to a method for detecting POMC accumulated in the cytosol of POMC neurons, including inducing an antigen-antibody reaction by treating a sample with an antibody or antigen-binding fragment of the present disclosure.

As used herein, the term “sample” refers to a biological sample obtained from a subject or patient. Sources of the biological sample may be fresh, frozen and/or preserved organ or tissue samples or solid tissues from biopsies or aspirates; blood or any blood components; and cells at any time point of conception or development in a subject.

In one aspect, the present disclosure relates to a kit for diagnosing metabolic diseases, including a POMC-specific antibody or antigen-binding fragment of the present disclosure.

In an embodiment, the kit may further include not only tools and/or reagents for collecting a biological sample from a subject or patient, but also tools and/or reagents for producing POMC from the sample.

In one aspect, the present disclosure relates to a pharmaceutical composition for preventing or treating metabolic diseases, including a Marchf6 protein or fragment thereof, or an activator or expression promoter of Marchf6 as an active ingredient.

In an embodiment, the fragment of Marchf6 may include a C4 domain including amino acids at positions 443 to 480 of the Marchf6 protein.

In an embodiment, the fragment of Marchf6 may include C9, an amino acid at position 9, and P460, an amino acid at position 460 in an amino acid sequence.

In an embodiment, the Marchf6 protein may be a variant or analog thereof, and the variant or analog may be a functional equivalent that retains the activity of degrading POMC.

In an embodiment, the Marchf6 protein may inhibit the degradation of Gpx4.

In an embodiment, the Marchf6 protein may prevent lipid peroxidation.

In an embodiment, the Marchf6 protein or fragment thereof may include an additional amino acid sequence designed for a specific purpose of increasing a targeting sequence, a tag, a labeled residue, and half-life or stability of the protein, and may further include a conjugated peptide/protein capable of binding to POMC neurons of the hypothalamus.

In an embodiment, the Marchf6 protein or fragment thereof may be linked to a coupling partner, such as an effector, a drug, a prodrug, a toxin, a peptide, a delivery molecule, or the like.

In an embodiment, the activator or expression promoter of Marchf6 may be a compound, a peptide, an aptamer, a primer, a probe or an antibody that specifically binds to the Marchf6 protein or a gene encoding the Marchf6 protein.

In an embodiment, the expression promoter may be a recombinant vector including a nucleic acid encoding Marchf6 or a fragment thereof.

In an embodiment, the activated or expression-promoted Marchf6, increased by the Marchf6 protein or fragment thereof, or the activator or expression promoter of Marchf6, may remove SP-uncleaved POMC that fails to be translocated into ER in the cytosol.

In an embodiment, Marchf6 degrades SP-containing POMC in the cytosol of POMC neurons, and Marchf6 regulates translocation of POMC into the ER by inhibiting lipid peroxidation.

In an embodiment, the recombinant vector may further include a POMC neuron-targeting ligand of the hypothalamus.

In an embodiment, the recombinant vector may further include a transcriptional regulator, a translational regulator or a marker capable of determining gene expression.

In an embodiment, the marker may be an antibiotic resistance gene, a selection marker gene, a P glucuronidase encoding gene, chloramphenicol acetyltransferase, luciferase, or a fluorescent protein encoding gene.

In an embodiment, the selection marker gene may be selected from the group consisting of neomycin phosphotransferase, hygromycin phosphotransferase, puromycin, histidinol dehydrogenase, guanine phosphotransferase, and zeocin, and more preferably a hygromycin phosphotransferase (htpII) gene.

In an embodiment, the fluorescent protein may be green fluorescent protein (GFP), enhanced green fluorescent protein (EGFP), yellow fluorescent protein (YFP), red fluorescent protein (RFP), orange fluorescent protein (OFP), cyan fluorescent protein (CFP), blue fluorescent protein (BFP), far-red fluorescent protein, or a tetracysteine motif.

In an embodiment, the recombinant vector may include a tag sequence, and the tag may be a His tag, a Myc (c-myc) tag, a FLAG tag, an HA tag, or a T7 tag.

In an embodiment, the composition may further include an activator or expression promoter of Bag6, Derl1 or VCP.

In an embodiment, the metabolic diseases may be any one selected from the group consisting of obesity, bulimia, diabetes, arteriosclerosis, hypertension, hyperlipidemia, fatty liver, metabolic liver disease, and cardiovascular disease, preferably obesity, and more preferably obesity caused by a hypothalamic disorder.

In an embodiment, the metabolic diseases may be obesity caused by ER stress or ferroptosis caused by a POMC protein accumulated in the cytosol of POMC neurons in the hypothalamus, and the obesity may have symptoms of bulimia, increased appetite, and decreased energy metabolism.

As used herein, the term “variant” refers to a corresponding amino acid sequence that contains at least one amino acid difference (substitution, insertion or deletion) compared to a reference substance. In specific embodiments, the “variant” has high amino acid sequence homology and/or conservative amino acid substitution, deletion and/or insertion compared to the reference sequence.

The protein variant according to the present disclosure is interpreted to include a variant in which an amino acid residue is conservatively substituted at a specific amino acid residue position.

As used herein, the “conservative substitution” means a modification of a variant that includes substituting one or more amino acids with amino acids having similar biochemical properties that do not cause loss of biological or biochemical functions of the corresponding Marchf6 protein or fragment thereof. The “conservative amino acid substitution” is a substitution that replaces an amino acid residue with an amino acid residue having a similar side chain. The kinds of amino acid residues having similar side chains are defined in the art and are well known. These kinds include amino acids having basic side chains (e.g., lysine, arginine, histidine), amino acids having acidic side chains (e.g., aspartic acid, glutamic acid), amino acids having uncharged polar side chains (e.g., glycine, asparagine, glutamine, serine, threonine, tyrosine, cysteine), amino acids having non-polar side chains (e.g., alanine, valine, leucine, isoleucine, proline, phenylalanine, methionine, tryptophan), amino acids having beta-branched side chains (e.g., threonine, valine, isoleucine), and amino acids having aromatic side chains (e.g., tyrosine, phenylalanine, tryptophan, histidine).

As used herein, the term “analog” may include protein analogs in which the side chains or the alpha-amino acid backbone of amino acids are substituted with one or more other functional groups. Examples of side chain or backbone modified peptide analogs may include hydroxyproline in which a pyrrolidine ring is substituted with a hydroxy group or N-methyl glycine “peptoids,” but are not limited thereto. The types of protein/peptide analogs are well known in the art.

As used herein, the term “nucleic acid” refers to deoxyribonucleotide or ribonucleotide that exists in a single-stranded or double-stranded form, and includes natural nucleic acid analogs unless specifically stated otherwise (Scheit, Nucleotide Analogs, John Wiley, New York (1980); Uhlman and Peyman, Chemical Reviews, 90:543584-584(1990)).

As used herein, the “expression promoter” means a substance that directly or indirectly acts on Marchf6 to improve, induce, stimulate, or increase the expression of Marchf6, and there is no limitation on the type of the substance. The mechanism in which the substance promotes the expression of Marchf6 is not particularly limited, and for example, the mechanism may act as a mechanism to increase gene expression such as transcription or translation, or to convert an inactive form into an active form.

As used herein, the “recombination vector” refers to a general term for a vector produced so as to express Marchf6, and preferably, includes a liposome, a plasmid vector, a cosmid vector, a bacteriophage vector, a viral vector, etc., and is not limited thereto as long as the vector is any vector capable of expressing Marchf6 in vivo. Examples of the viral vectors include adenovirus, adeno-associated virus, retrovirus, lentivirus, herpes simplex virus, alpha virus, etc.

The pharmaceutical composition of the present disclosure may further include a known metabolic disease therapeutic agent as the active ingredient, in addition to the Marchf6 protein or fragment thereof, or the activator or expression promoter of Marchf6, and may be used in combination with other treatments known for the treatment of these diseases.

As used herein, the term “prevention” means all actions of inhibiting or delaying the occurrence, spread, and recurrence of the metabolic disease by administering the pharmaceutical composition according to the present disclosure, and the “treatment” means all actions of improving or beneficially changing the symptoms of the metabolic disease by administering the composition of the present disclosure. Those skilled in the art to which the present disclosure pertains will be able to determine the degree of improvement, enhancement and treatment by knowing the exact criteria of the disease for which the composition of the present disclosure is effective by referring to data presented by the Korean Medical Association, etc.

As used herein, the term “therapeutically effective amount” used in combination with the active ingredient means an amount effective to prevent or treat a target disease, and the therapeutically effective amount of the composition of the present disclosure may vary depending on several factors, such as a method of administration, a target site, the condition of a patient, etc. Accordingly, when used in the human body, a dose should be determined as an appropriate amount in consideration of both safety and efficiency. It is also possible to estimate an amount to be used in humans from the effective amount determined through animal experiments. These matters to be considered when determining the effective amount are described in, for example, Hardman and Limbird, eds., Goodman and Gilman's The Pharmacological Basis of Therapeutics, 10th ed. (2001), Pergamon Press; and E. W. Martin ed., Remington's Pharmaceutical Sciences, 18th ed. (1990), Mack Publishing Co.

The pharmaceutical composition of the present disclosure is administered in a pharmaceutically effective amount. As used herein, the term “pharmaceutically effective amount” refers to an amount enough to treat the disease at a reasonable benefit/risk ratio applicable to medical treatment and not to cause side effects. The effective amount level may be determined according to factors including the health condition of a patient, the type of metabolic disease, the cause and severity of metabolic disease, the activity of a drug, the sensitivity to a drug, a method of administration, a time of administration, a route of administration, an excretion rate, duration of treatment, and drugs used in combination or concurrently, and other factors well-known in the medical field. The composition of the present disclosure may be administered as an individual therapeutic agent or in combination with other therapeutic agents, and may be administered sequentially or simultaneously with conventional therapeutic agents, and may be administered as a single dose or in multiple doses. It is important to administer an amount capable of obtaining a maximum effect with a minimal amount without side effects by considering all the factors, which may be easily determined by those skilled in the art.

The pharmaceutical composition of the present disclosure may include carriers, diluents, excipients, or a combination of two or more thereof, which are commonly used in biological agents. As used herein, the term “pharmaceutically acceptable” means that the composition exhibits non-toxic properties to cells or humans exposed to the composition. The carrier is not particularly limited as long as the carrier is suitable for in vivo delivery of the composition, and may be used by combining, for example, compounds described in Merck Index, 13th ed., Merck & Co. Inc., saline, sterile water, a Ringer's solution, buffered saline, a dextrose solution, a maltodextrin solution, glycerol, ethanol, and one or more of these components, and if necessary, other conventional additives such as an antioxidant, a buffer, and a bacteriostat may be added. In addition, the pharmaceutical composition may be formulated in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be formulated preferably according to each disease or ingredient using a suitable method in the art or a method disclosed in Remington's Pharmaceutical Science (Mack Publishing Company, Easton PA, 18th, 1990).

In an embodiment, the pharmaceutical composition may be one or more formulations selected from the group consisting of oral formulations, external preparations, suppositories, sterile injections and sprays, and more preferably oral or injectable formulations.

As used herein, the term “administration” means providing a predetermined substance to a subject or patient by any suitable method, and the pharmaceutical composition may be administered parenterally (e.g., applied as an injectable formulation intravenously, subcutaneously, intraperitoneally or topically) or orally according to a desired method. The dose range may vary depending on the body weight, age, sex, and health condition of a patient, a diet, an administration time, an administration method, an excretion rate, the severity of a disease, etc. Liquid formulations for oral administration of the composition of the present disclosure correspond to suspensions, internal solutions, emulsions, syrups, etc., and may include various excipients, such as wetting agents, sweeteners, fragrances, preservatives, and the like, in addition to water and liquid paraffin, which are commonly used simple diluents. Formulations for parenteral administration include sterilized aqueous solutions, non-aqueous solvents, suspensions, emulsions, lyophilized agents, suppositories, and the like. The pharmaceutical composition of the present disclosure may also be administered by any device capable of translocating an active substance to a target cell. Preferred administration methods and formulations are intravenous injections, subcutaneous injections, intradermal injections, intramuscular injections, drop injections, etc. The injections may be prepared by using aqueous solvents such as a physiological saline solution and a Ringer's solution, and non-aqueous solvents such as vegetable oils, higher fatty acid esters (e.g., ethyl oleate), and alcohols (e.g., ethanol, benzyl alcohol, propylene glycol, or glycerin). The injections may include pharmaceutical carriers, such as a stabilizer (e.g., ascorbic acid, sodium hydrogen sulfite, sodium pyrosulfite, BHA, tocopherol, EDTA, etc.) for the prevention of degeneration, an emulsifier, a buffer for pH control, and a preservative (e.g., phenyl mercury nitrate, thimerosal, benzalkonium chloride, phenol, cresol, benzyl alcohol, etc.) to inhibit microbial growth.

As used herein, the term “subject” refers to all animals including monkey, cow, horse, sheep, pig, chicken, turkey, quail, cat, dog, mice, rats, rabbit or guinea pig including humans who have developed or may develop the metabolic diseases, and the pharmaceutical composition of the present disclosure may be administered to a subject to effectively prevent or treat the diseases.

The pharmaceutical composition of the present disclosure may be administered in combination with existing therapeutic agents.

The pharmaceutical composition of the present disclosure may further include pharmaceutically acceptable additives. At this time, the pharmaceutically acceptable additives may use starch, gelatinized starch, microcrystalline cellulose, lactose, povidone, colloidal silicon dioxide, calcium hydrogen phosphate, lactose, mannitol, syrup, arabic gum, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, white sugar, dextrose, sorbitol, talc and the like. The pharmaceutically acceptable additive according to the present disclosure is preferably included in an amount of 0.1 part by weight to 90 parts by weight based on the composition, but is not limited thereto.

The pharmaceutical composition of the present disclosure may be used as a single therapy, but may also be used in combination with other conventional biological therapy or chemotherapy, and may treat more effectively metabolic diseases in the case of such a concurrent therapy.

The present disclosure provides a method for preventing or treating diseases associated with membrane associated ring-CH-type finger 6 (Marchf6) dysfunction or metabolic diseases, including administering the pharmaceutical composition to a subject in a pharmaceutically effective amount.

In one aspect, the present disclosure relates to a composition for promoting the degradation of pro-opiomelanocortin (POMC), including a Marchf6 protein or fragment thereof, or an activator or expression promoter of Marchf6.

In an embodiment, the composition may promote the degradation of POMC in the cytosol of POMC neurons.

In an embodiment, the fragment of Marchf6 may include a C4 domain including amino acids at positions 443 to 480 of the Marchf6 protein.

In an embodiment, the fragment of Marchf6 may include C9, an amino acid at position 9, and P460, an amino acid at position 460 in an amino acid sequence.

In an embodiment, the expression promoter may be a recombinant vector including a nucleic acid encoding Marchf6 or fragment thereof.

In an embodiment, the P460 site of Marchf6 recognizes a site containing a signal peptide (SP) of POMC as a degron to induce the degradation of POMC.

In one aspect, the present disclosure relates to a composition for promoting translocation into ER, including a Marchf6 protein or fragment thereof, or an activator or expression promoter of Marchf6.

In one aspect, the present disclosure relates to a composition for inhibiting ER stress or ferroptosis in POMC neurons, including a Marchf6 protein or fragment thereof, or an activator or expression promoter of Marchf6.

In an embodiment, the composition may inhibit increased ER stress or ferroptosis induced by excess POMC or cytosolic remaining POMC.

In an embodiment, the P460 site of Marchf6 in the cytosol of POMC neurons may recognize a site containing the signal peptide (SP) of POMC as a degron to degrade POMC and translocate POMC into ER, thereby preventing/inhibiting ER stress and ferroptosis.

In one aspect, the present disclosure relates to a food composition for preventing or improving metabolic diseases, including a Marchf6 protein or fragment thereof, or an activator or expression promoter of Marchf6 as an active ingredient.

When the composition of the present disclosure is used as the food composition, the Marchf6 protein or fragment thereof, or the activator or expression promoter of Marchf6 may be added as it is or used together with other foods or food ingredients, and may be used appropriately according to a conventional method. The composition may include food acceptable supplement additives in addition to the active ingredients, and the mixing amount of the active ingredients may be appropriately determined depending on the purpose of use (prevention, health or therapeutic treatment).

As used herein, the term “food supplement additive” means a component that may be supplementally added to food, and may be appropriately selected and used by those skilled in the art as additives to be added to prepare a health functional food of each formulation. Examples of the food supplement additive include various nutrients, vitamins, minerals (electrolytes), flavors such as synthetic and natural flavors, colorants and fillers, pectic acid and salts thereof, alginic acid and salts thereof, organic acids, protective colloidal thickeners, pH adjusters, stabilizers, preservatives, glycerin, alcohols, carbonating agents used in carbonated drinks, and the like, but the types of food supplement additive of the present disclosure are not limited to the examples.

The food composition of the present disclosure may include a health functional food. As used herein, the term “health functional food” refers to food prepared and processed in the form of tablets, capsules, powders, granules, liquids and pills by using raw materials or ingredients having functionality useful to the human body. Here, the ‘functionality’ means regulating nutrients to the structure and function of the human body or obtaining effects useful for health applications such as physiological action. The health functional food of the present disclosure is able to be prepared by methods commonly used in the art and may be prepared by adding raw materials and ingredients which are commonly added in the art during the preparation. In addition, the formulation of the health functional food may also be prepared with any formulation recognized as a health functional food without limitation. The food composition of the present disclosure may be prepared in various forms of formulations, and the health functional food of the present disclosure may be consumed as a supplement to enhance the effect of a metabolic disease therapeutic agent.

In addition, there is no limitation in the types of health food in which the composition of the present disclosure may be used. In addition, the composition including the Marchf6 protein or fragment thereof, or the activator or expression promoter of Marchf6 of the present disclosure as the active ingredient may be prepared by mixing other appropriate auxiliary ingredients that may be contained in a health functional food and known additives according to the selection of those skilled in the art. Examples of food which may be added include meat, sausage, bread, chocolate, candy, snacks, confectionery, pizza, ramen, other noodles, gum, dairy products including ice cream, various soups, beverages, tea, drinks, alcoholic beverages, vitamin complexes and the like, and may be prepared to be added to extract, tea, jelly, juice, and the like prepared by using the extract according to the present disclosure as a main ingredient.

In one aspect, the present disclosure relates to a method for screening an obesity therapeutic agent, including treating cells isolated from a subject with a candidate substance; determining an expression or activation level of Marchf6 in the cells treated with the candidate substance; and comparing the expression or activation level of Marchf6 with that of a control group.

In one embodiment, the cells may be POMC neurons.

### [Modes]

Hereinafter, the present disclosure will be described in more detail through the following Examples. However, the following Examples are only intended to embody the contents of the present disclosure, and the present disclosure is not limited thereto.

### Example 1. Search for Proteins Interacting with Marchf6

To investigate proteins binding to membrane associated ring-CH-type finger 6 (Marchf6), a yeast two-hybrid (Y2H) assay was performed using eight cytosolic-facing regions (C1 to C8) of Marchf6. Specifically, a human cDNA library (638820, Clontech) as bait was transformed into S. cerevisiae AH109 (CHY726) cells expressing plasmids pCH4537 (Marchf6 C1 fragment expression), pCH4538 (Marchf6 C2 fragment expression), pCH4539 (Marchf6 C3 fragment expression), pCH4540 (Marchf6 C4 fragment expression), pCH4541 (Marchf6 C5 fragment expression), pCH4542 (Marchf6 C6 fragment expression), pCH4543 (Marchf6 C7 fragment expression), and pCH4544 (Marchf6 C8 fragment expression) (FIG. 1). Among ~1×10⁶ transformants, 14 library plasmids were recovered from ~100 positive clones and analyzed by DNA sequencing to select 9 plasmids having specific human genes (FIG. 1B). In addition, in order to further confirm the interaction of molecules detected to bind to each domain of Marchf6 with full-length Marchf6, split-Ub assay was performed by co-transforming CHY712 S. cerevisiae cells with pCH836 (Marchf6) as bait and pCH4203 (POMC), pCH7140 (Bnip3), pCH7141 (Gps1), pCH7142 (SNAPIN), pCH7143 (Yifla), pCH7144 (Hspa5), pCH7145 (Hax1), pCH7146 (Fhl2), and pCH7147 (Mcfd2) as preys. The produced transformants were grown to A₆₀₀=1, serially diluted 5-fold, spotted on a SC(-Leu/-Trp) or SC(-Leu/-Trp/-His) plate, and incubated at 30° C. for 3 days. Among these transformants, only yeast cells having pCH836 (Marchf6) and pCH4203 (POMC) survived on the SC plate (-Leu/-Trp/-His), and then it was confirmed that POMC bound to full-length Marchf6 (FIG. 1C), indicating an interaction between Marchf6 and POMC.

### Example 2. Search for Interaction Sites of Marchf6 and POMC

### 2-1. Y2H Assay and Split-Ub Assay

To determine which residue in the C4 domain of Marchf6 interacting with POMC in Example 1 was important for interacting with POMC, pCH4540 (C4) and amino acid substitution variants of the conserved region thereof, pCH4158 (C^{R443A}), pCH4159 (C^{R447A}), pCH4160 (CA^{K451A}), pCH4161 (C^{4D459A}), pCH4162 (C^{P460A}), and pCH4163 (C^{R479A}), were co-transformed into CHY726 together with pCH4132 (POMC), respectively. Transformants including the generated bait and prey plasmids were spotted on the SC plate and incubated at 30° C. for 3 days.

As a result, in the C4 domain of Marchf6 specifically binding to POMC, only mutation P460A (Pro460-to-Ala) was found to lose binding affinity, unlike mutations R443A (Arg443-to-Ala), R447A (Arg447-to-Ala), L451A (Leu451-to-Ala), D459A (Asp459-to-Ala), and R479A (Arg479-to-Ala) (FIGS. 2A to 2C).

### 2-2. Chemical Crosslinking-Based Reciprocal Co-Immunoprecipitation Assay

For the chemical crosslinking-based reciprocal co-immunoprecipitation assay, HEK293T cells were incubated at −70% confluency in a 10-cm dish plate and co-transfected with 2 g of pCH60 (empty vector) or pCH4129 (exogenous C-terminally hemagglutinin (ha)-tagged POMC: POMCₕₐ) and pCH879 (wild-type C-terminal triple flag-tagged Marchf6: Marchf63f) or pCH4170 (Marchf6^{P460A}) and incubated for 48 hours. The cells were washed with PBS, treated with 1 mM DSP [dithiobis(succinimidyl propionate)] (Thermo Fisher, 22585), and then the crosslinked cells were lysed and immunoprecipitated. The beads were washed three times with an IP-wash buffer (20 mM Tris-HCl, 137 mM NaCl, 0.1% NP-40, 2 mM EDTA, 10% glycerol) and bound proteins were eluted with a 2× SDS sample buffer. For immunoprecipitation with FLAG-agarose (Sigma-Aldrich, A2220), a 3× FLAG peptide (Protein Ark, GEN-3XFLAG-5) was used. Thereafter, the eluate was denatured and heated at 37° C. for 20 minutes, and SDS-PAGE and immunoblotting were performed using the corresponding antibody.

As a result, it was found that full-length POMC specifically bound to Marchf6, but did not bind to Marchf6^{P460A} (FIGS. 2D and E), confirming that POMC bound to P460 of the C4 domain of Marchf6.

### Example 3. Confirmation of POMC Degradation Through Interaction Between Marchf6 and POMC

### 3-1. Analysis of POMC Degradation Mechanism of Marchf6

To determine proteasomal degradation of POMC due to specific recognition for POMC by Marchf6 Ub ligase, cycloheximide (CHX)-chase assay of protein degradation and ubiquitylation assay were performed. Specifically, for the CHX-chase assay, Marchf6-KO N43/5 cells were constructed using the CRISPR/Cas9 system using sgRNA targeting exon 6 of the Marchf6 locus, dispensed at a density of 1×10⁵ cells, incubated for 24 hours, and treated with CHX at a final concentration of 50 μg/ml. Thereafter, the cells were harvested at each time and lysed with RIPA buffer (Thermo Fisher Scientific, 89900) containing a 1× protease inhibitor cocktail (Sigma-Aldrich, 4693132001) on ice for 20 minutes, and then centrifuged at 16,500×g for 20 minutes at 4° C. to collect the supernatant. The protein concentration in the supernatant was measured by Bradford assay (Bio-Rad, 5000006), and an equal amount of protein was separated by SDS-PAGE and immunoblotted with the corresponding antibody. Quantification of immunoblotting data was performed by GelQuant.NET (BiochemLabSolutions) and standardized based on α-tubulin. In addition, an immunoblotting assay of POMCₕₐ and Marchf6_{3f} was performed in HEK293T cells co-expressing POMCₕₐ and/or Marchf6_{3f} incubated in the presence or absence of MG132 (10 M) for 8 hours. In addition, for the ubiquitination assay, HEK293T cells were transfected with POMC_{myc}, ^{ha}Ub, and Marchf63f or Marchf6_{3fP46}% incubated for 48 hours, and then treated or untreated with 10 μM MG132 for 8 hours. Thereafter, the cells were lysed with a lysis buffer (50 mM Tris-HCl, 150 mM NaCl, 1 mM EDTA, 1% NP-40, pH 7.6) containing 10 μM N-ethylmaleimide (NEM) (Sigma-Aldrich, E3876) and 1× protease inhibitor cocktail, and the supernatant was immunoprecipitated with magnetic bead-conjugated rabbit anti-myc (GeneTex, GTX29106). The bound protein was eluted from the magnetic beads with a 2× SDS sample buffer, the sample was heated at 95° C. for 5 minutes, and the eluted protein was separated using SDS-PAGE and immunoblotted with the corresponding antibody.

As the CHX-chase assay result, it was found that exogenous POMCₕₐ was short-lived in WT HEK293T (human embryonic kidney) cells, but significantly stabilized in Marchf6-KO cells (FIGS. 3A and 3B), and endogenous POMC was also short-lived in N43/5 (mouse embryonic hypothalamus) cells, but significantly stabilized in Marchf6-KO N43/5 cells (FIG. 3C). In addition, the endogenous POMC stabilized in Marchf6-KO N43/5 cells was short-lived when Marchf63f was expressed with three flags tagged at the C-terminus of WT Marchf6, and POMC degradation did not occur when Marchf6_{3f}^{P460A}, which had a mutation introduced into the POMC-interacting region, was expressed. Squalene monooxygenase (SM), known as a substrate of Marchf6, was also found to be degraded even when Marchf6_{3f}^{P460A} was expressed (FIG. 2F), confirming that Marchf6 degraded POMC by direct binding of the P460 site of Marchf6 to POMC.

In addition, as the immunoblotting assay result of POMCₕₐ and Marchf6_{3f}, it was found that the level of POMCₕₐ was reduced by co-expression of Marchf63f and POMCₕₐ, which was restored by treatment with the proteasome inhibitor MG132 (FIG. 2G), and thus it was confirmed that the degradation of POMCₕₐ was proteasome-dependent.

Furthermore, as the ubiquitination assay result in cells expressing POMC_{myc} (C-terminally myc-tagged POMC) together with ^{ha}Ub (N-terminally ha-tagged Ub), it was found that polyubiquitinated POMC_{myc} was increased by co-expression with Marchf6_{3f}, whereas when co-expressing POMC-inaccessible Marchf6_{3f}^{P460} or catalytically inactive Marchf6_{3f}^{C9A}, the polyubiquitinated POMC_{myc} was not increased (FIGS. 2H and 3D).

### 3-2. Confirmation of Association Between POMC Degradation and ERpQC Pathway

To determine whether a stress-induced pre-emptive protein quality control (ERpQC) pathway was involved in Marchf6-mediated POMC degradation, HEK293T cells were transfected with 15 nM siRNAs (Bag6: SCBT, 15 sc72614; Derl1: SCBT, sc-60519; VCP: SCBT, sc-37187) against Bag6 (chaperone), Derl1 (ER recruiting factor) and VCP (a valosincontaining AAA+-ATPase; also known as p97), which were key components of ERpQC using Lipofectamine RNAiMAX (Thermo Fisher Scientific, 13778150), incubated for 24 hours, and then transfected with 0.5 μg/ml of pCH4129 (POMCₕₐ) using Lipofectamine 2000 (Thermo Fisher Scientific, 11668019) and incubated for 24 hours. Thereafter, the CHX-chase assay was performed.

As a result, genes in the ERpQC pathway knocked down with siRNA were found to stabilize POMCₕₐ (FIG. 3E) without affecting the level of POMC mRNA (FIG. 3F), confirming that the ERpQC pathway was positively involved in the degradation of cytosol-exposed POMC.

### Example 4. Specificity of POMC Target Site of Marchf6

To clarify a POMC domain binding to the C4 domain of Marchf6, a Y2H assay was performed using a set of POMC fragments in which the C4 of Marchf6 and the C-terminus (Ct-) of POMC were cleaved. To this end, pCH4540 (C4), pCH4132 (POMC), or cleaved forms thereof pCH4235 (POMC¹⁻⁷⁶), pCH4236 (POMC¹⁻⁸⁷), pCH4237 (POMC¹⁻¹⁰²), pCH4238 (POMC¹⁻¹³⁷), and pCH4239 (POMC¹⁻¹⁷⁶) were co-transformed into CHY726 and spotted on a SC(-Leu/-Trp) or SC(-Leu/-Trp/-His) plate and analyzed as in Example above. In addition, for interaction analysis between full-length Marchf6 and POMC, a split-Ub assay was performed by transforming S. cerevisiae CHY712 with pCH836 (Marchf6) or pCH4311 (Marchf6^{P460A}) as bait and pCH4203 (POMC¹⁻²⁶⁷), pCH4204 (POMC⁷⁷⁻²⁶⁷), or pCH4313 (POMC¹⁻⁷⁶) as prey. In addition, for glutathione-S-transferase (GST) pull-down assay, 50 g of purified GST, GST-POMC¹⁻⁷⁶, GST-POMC²⁷⁻⁷⁶, or GST-POMC¹⁻²⁶ was incubated with 50 μl of glutathione Sepharose beads (Cytiva, 17-0756-05) at 4° C. for 2 hours.

HEK293T cells transfected with pCH879 (Marchf6_{3f}) were lysed with a lysis buffer (50 mM Tris-HCl, 150 mM NaCl, 1 mM EDTA, 1% NP-40, pH 7.6) containing 1× protease inhibitor cocktail to obtain an extract, and the extract and the beads were further incubated at 4° C. for 2 hours, and then washed three times with 0.5 ml of GST-wash buffer (20 mM Tris-HCl, 137 mM NaCl, 2 mM EDTA, 0.5% NP-40, 10% glycerol, pH 7.6). Proteins binding to the beads were eluted with a 2× SDS-sample buffer, heated at 37° C. for 20 minutes, subjected to SDS-PAGE, Coomassie blue staining, and immunoblotting using anti-flag antibody.

As a result of analyzing a POMC recognition site by Marchf6 using Y2H, it was found that the C4 domain of Marchf6 interacted with POMC¹⁻⁷⁶, which included the signal peptide (SP) and the Nt-segment of the Nt-POMC domain (N-POMC) (FIG. 4A). In addition, as the split-Ub assay result, it was found that Marchf6 interacted with POMC¹⁻²⁶⁷ and POMC¹⁻⁷⁶, but did not interact with POMC⁷⁷⁻²⁶⁷ (FIG. 4B). In addition, as the GST pull-down assay result, it was found that Marchf63f directly bound to GST-POMC¹⁻⁷⁶, very weakly bound to GST-POMC²⁷⁻⁷⁶, and hardly bound to GST-POMC¹⁻²⁶ (FIG. 4C). Through this, it was confirmed that Marchf6 recognized a degron that included SP and was adjacent to the Nt-POMC segment.

### Example 5. Regulation of POMC Translocation of Marchf6 into ER

### 5-1. Production of SP-Containing POMC-Specific Antibodies

To produce SP-containing POMC-specific antibodies, a peptide (SEQ ID NO: 1) of QASMEVRGWC (Q, Gln; A, Ala; S, Ser; M, Met; E, Glu; V, Val; R, Arg; W, Trp; and C, Cys) corresponding to positions 19 to 28 of human POMC was synthesized as an epitope (FIG. 4D), and rabbit polyclonal antisera against SP-containing POMC-derived peptides were produced by AbClon (Seoul, South Korea). Thereafter, SP-containing POMC-specific antibodies were “negatively” selected from 1 ml of antiserum incubated overnight at 4° C. with 1 ml of Affi-Gel 10/15 beads containing 1.5 mg of pre-conjugated GST-POMC²⁷⁻⁷⁶, and the flow-through fractions were further incubated overnight at 4° C. with 1 ml of Affi-Gel 10/15 beads containing 1 mg of pre-conjugated GST-POMC¹⁻⁷⁶. Thereafter, the beads were washed at least five times with 10 ml of ice-cold PBS, and the antibodies binding to the beads were eluted with 1 ml of 0.1 M glycine-HCl (pH 3.0). The eluted fraction was immediately neutralized with 1 M Tris (pH 8.0) and concentrated using protein A-Sepharose chromatography (Amicogen, Korea) to produce SP-containing POMC-specific antibodies, which were designated as anti-POMC^{SP} antibodies. To confirm whether the antibodies produced in this way were able to detect SP-containing POMC, an immunoblotting assay was performed using GST-POMC¹⁻⁷⁶, GST-POMC²⁷⁻⁷⁶ (without SP), and GST-POMC¹⁻²⁶ (containing SP).

As a result, the anti-POMC^{SP} antibodies specifically detected GST-POMC¹⁻⁷⁶ and GST-POMC¹⁻²⁶ containing SP, but did not recognize GST-POMC²⁷⁻⁷⁶ without SP (FIG. 4E).

### 5-2. Confirmation of Regulation of POMC Intracellular Translocation by Marchf6

To confirm the degradation of POMC by Marchf6 and resulting subcellular localization, WT HEK293T cells or Marchf6-KO HEK293T cells were transfected with pCH4129 (POMCₕₐ), and then treated with MG132 (10 M) for 8 hours in the presence or absence of Eeyarestatin I (EerI) (8 μM), an ER translocation inhibitor, and subcellular fractionation was performed for cytosol-organelle fractionation assay, followed by immunoprecipitation (IP)-immunoblotting (IB) assay (FIG. 4F). Specifically, the cells were washed with PBS, treated with semi-permeabilization buffer containing 1× protease inhibitor cocktail (110 mM KOAc, 50 mM HEPES, 2 mM MgCl₂, 1 mM benzamidine, 0.01% digitonin, pH 7.4) on ice for 5 minutes, and centrifuged at 500×g for 10 minutes at 4° C. to obtain the supernatant as the cytosolic fraction. Thereafter, after separating the supernatant, the cells were washed with cold PBS to remove a remaining digitonin extract, and lysed with an IP buffer (50 mM HEPES, 150 mM NaCl, 1% Triton X-100, pH 7.4) containing 1× protease inhibitor cocktail on ice for 10 minutes. After lysis, the cells were centrifuged at 12,000×g for 10 minutes at 4° C., and the supernatant was obtained as the organelle fraction. The cytosolic and organelle fractions were incubated overnight at 4° C. with the anti-POMC^{SP} antibody (1:2,000) prepared in Example 5-1, and added with Dynabeads Protein G (Thermo Fisher Scientific, 10004D) and incubated for an additional 90 minutes at 4° C. To remove non-specifically binding or unbinding proteins, the cytosolic and organelle fractions were washed three times with an IP washing solution (50 mM HEPES, 150 mM NaCl, 0.5% Triton X-100, pH 7.4) for 5 minutes each at 4° C., and the immunoprecipitated proteins were eluted with a 2× SDS-sample buffer, heated at 95° C. for 5 minutes, and subjected to SDS-PAGE, and immunoblotting assay using anti-HA.

As a result, POMCₕₐ containing SP was detected in the cytosolic fractions of Marchf6-KO HEK293T cells, but not detected in wild-type cells (FIG. 4G, cf. Lanes 5 and 7). In particular, in the case of treatment with EerI, an ER translocation inhibitor, not only SP-containing POMCₕₐ was increased in the cytosolic fraction (digitonin-extracted) of Marchf6-KO HEK293T cells, but also the prohormone was detectable in wild-type cells (FIG. 4G, Lanes 5 to 7).

Through these results, it was confirmed that Marchf6 positively regulated the translocation and degradation of early POMC into ER.

### Example 6. Confirmation of POMC-Induced ER Stress and Ferroptosis Inhibition Effects of Marchf6

### 6-1. Induction of ER Stress and Ferroptosis by Excessive POMC

To understand the functional or mechanistic association between Marchf6-mediated POMC degradation and ferroptosis, lipid ROS levels were compared in N43/5 cells overexpressing POMCₕₐ using a lipid peroxidation sensor C11-BODIPY^{581/591}. Specifically, WT or Marchf6-KO N43/5 cells were transfected with pCH4129 (POMCₕₐ) for 48 hours, and the medium was replaced with a culture medium containing 2 μM C11-BODIPY^{581/591} (Thermo Fisher Scientific, D3861). The cells were incubated at 37° C. for 30 minutes, and the fluorescent probe-treated cells were trypsinized, transferred to a 15 ml Falcon tube, washed three times with 1 ml of ice-cold PBS, and then transferred to an ice-cold round-bottom polystyrene tube (Corning, 352235). The amount of intracellular lipid ROS in −20,000 cells was measured using CytoFLEX LX (Beckman Coulter) using fluorescein isothiocyanate (FITC) and analyzed using FlowJo v10.8.1 (BD Bioscience). In addition, cell viability was analyzed using a CellTiter-Glo luminescent 3D cell viability assay kit (Promega, G9241) and a multimode plate reader (TECAN, Spark 10 M). At this time, when the inhibition of cell death was evaluated, WT or Marchf6-KO N43/5 cells expressing pCH60 (empty vector) or pCH4129 (POMCₕₐ) (final concentration of 1 g/ml each) were treated with 5 μM Fer-1, 20 μM Z-VAD-FMK or 40 μM Nec-1, and incubated for 24 hours, and cell viability was analyzed. In addition, the intracellular NADP(H) level was measured using a NADP/NADPH-Glo assay kit (Promega, G9081) and a multimode plate reader (TECAN, Spark 10 M). Further, LDH activity was measured using the Cytotoxicity Detection Kit Plus (LDH) (Sigma, 4744926001).

As a lipid peroxidation assay result, it was found that as the POMCₕₐ level increased in N43/5 cells, lipid peroxidation increased (FIG. 5A), and the level of NADP(H), a biomarker capable of predicting ferroptosis, decreased (FIG. 5B). In addition, as the level of POMCₕₐ increased, cell viability gradually decreased (FIG. 5C), and dose-dependent lethality of POMCₕₐ was further confirmed by evaluating the release of lactate dehydrogenase (LDH) (FIG. 5D). In addition, the expression levels of ferroptosis-related proteins Gpx4, CHOP, Nox2, and Nox4 were analyzed by immunoblotting, and as a result, as the level of POMCₕₐ increased, the expression level of glutathione peroxidase 4 (Gpx4), a key regulator of ferroptosis, decreased, and the levels of a C/EBP homologous transcription factor (CHOP) as an ER stress marker, and Nox2 and Nox4, which were ROS-generating NADPH oxidases, also increased (FIGS. 5E and 8A). In addition, the overexpression of POMCₕₐ was found to significantly increase the level of 4-hydroxynonenal (4HNE) as a lipid peroxidation marker, which was restored by treatment with Fer-1, a lipid peroxide scavenger (FIG. 5F).

Through this, it was confirmed that overloaded POMC induced ferroptosis by downregulating Gpx4 and upregulating CHOP, Nox2, and Nox4 to increase lipid peroxidation.

### 6-2. POMC-Induced Ferroptosis Inhibition of Marchf6

Through Examples, since Marchf6 mediated the degradation of POMC, it was expected that loss of Marchf6 would make POMC neurons hypersensitive to POMC-induced ferroptosis. Therefore, pCH60 (empty vector), pCH879 (Marchf6_{3f}), pCH880 (Marchf6_{flag}^{C9A}), or pCH4170 (Marchf6_{flag}^{P460A}) was transfected into WT or Marchf6-KO N43/5 cells, and the degree of ferroptosis induction in POMC neurons was analyzed as in Example 6-1.

As a result, the viability of POMCₕₐ-overexpressing N43/5 cells was significantly reduced when Marchf6 was deficient, which was restored by ectopic expression of WT Marchf6_{3f}, but not restored by catalytically inactive Marchf6₃fc^{9A} and POMC-inaccessible Marchf6_{3f}^{P460A} (FIGS. 5G and 5H). In addition, the decreased expression levels of Gpx4, CHOP, Nox2, and Nox4 proteins in POMCₕₐ-overexpressing Marchf6-KO N43/5 cells were restored by Marchf63f, but not restored by Marchf6_{3f}^{C9A} and Marchf6_{3f}^{P460A} (FIG. 5I). In addition, it was found that the decreased viability of POMCₕₐ-overexpressing Marchf6-KO N43/5 cells was restored by treatment with a ferroptosis inhibitor, ferrostatin-1 (Fer-1), but not restored by treatment with an apoptosis inhibitor Z-VAD (Z-VAD-FMK) or Nec-1 (necroptosis inhibitor necrostatin-1) (FIG. 5J).

Through this, it was confirmed that Marchf6 inhibited ferroptosis in POMC neurons by specifically promoting POMC degradation.

### 6-3. Induction of Ferroptosis and ER Stress by POMC Remaining in Cytosol

To determine a biological effect of POMC remaining in the cytosol, which was not degraded by Marchf6, POMC^{Δ1-26}, which remained/maintained in the cytosol due to the absence of ER-targeting SP, was expressed in N43/5 POMC neurons, and cell viability was analyzed. The lipid peroxidation, NADP(H) content, LDH release, the expression levels of Gpx4, CHOP, Nox2, and Nox4, the expression of an ER stress marker CHOP, and the expression and activation levels of ER stress sensors such as protein kinase RNA-like ER kinase (PERK), inositol requiring protein-1 (Ire1), and activating transcription factor-6 (Atf6) were analyzed.

As a result, POMCₕₐ^{Δ1-26} without SP showed a greater effect on lipid peroxidation, NADP(H) content, LDH release, and cell viability than POMCₕₐ (FL, full length) containing SP (FIGS. 6A to 6D). In addition, the expression of cytosolic POMCₕₐ^{Δ1-26} in N43/5 cells significantly increased the levels of CHOP, Nox2, and Nox4, and significantly decreased the level of Gpx4 compared to expression of full-length POMCₕₐ (FIG. 6E). In addition, cytosolic POMCₕₐ^{Δ1-26} upregulated the expression of CHOP, an ER stress marker, and then the expression and activation due to phosphorylation were analyzed on upstream components of a signaling pathway related thereto, such as a eukaryotic initiation factor (eIF2α), PERK, Ire1, and Atf6. As a result, the overexpression of POMCₕₐ or POMCₕₐ^{Δ1-26} increased the phosphorylation of PERK and eIF2α, and up-regulated Atf4, but did not affect Ire1 and Atf6 (FIG. 6E), confirming that POMC remaining in the cytosol induced an ER stress response through a PERK/eIF2α/Atf4/CHOP signaling pathway.

### Example 7. Analysis of ER Stress and Ferroptosis-Inducing Mechanisms of Cytosolic POMC

### 7-1. Gpx4 Degradation Through Chaperone-Mediated Autophagy

To determine whether full-length POMCₕₐ or cytosolic POMCₕₐ^{Δ1-26} regulated the expression of Gpx4 protein, which was known to be degraded by ferroptosis induction, the mRNA level of Gpx4 was analyzed by qRT-PCR in N43/5 cells overexpressing full-length POMCₕₐ or cytosolic POMCₕₐ^{Δ1-26}, and subjected to CHX-chase assay. In addition, since Gpx4 degradation occurred through chaperone-mediated autophagy (CMA), macroautophagy, or the Ub-proteasome system, N43/5 cells overexpressing POMCₕₐ^{Δ1-26} were treated with a lysosomal inhibitor such as chloroquine (CQ) or BafA1 (bafilomycin A1), a proteasome inhibitor MG132, or a macroautophagy inhibitor 3-MA (3-methyladenine), and the expression of Gpx4 protein was analyzed by immunoblotting. In addition, the expression of lysosome-associated protein 2A (Lamp2a), heat shock chaperones Hsc70 and Hsp90 was confirmed by immunoblotting in N43/5 cells overexpressing POMCₕₐ^{Δ1-26}, and changes in Gpx4 were analyzed by treatment with Lamp2a-specific siRNA (SCBT, sc-35791).

As a result, the expression of full-length POMCₕₐ and cytosolic POMCₕₐ^{Δ1-26} downregulated protein expression without affecting the mRNA level of Gpx4. In particular, POMCₕₐ^{Δ1-26} remaining in the cytosol significantly downregulated Gpx4 compared to ER-translocable POMCₕₐ (FIGS. 6E and 6F), confirming that POMC in the cytosol may induce Gpx4 degradation. In addition, as the CHX-chase experiment result, it was found that cytosolic POMCₕₐ^{Δ1-26} accelerated the degradation of Gpx4 (FIG. 6G). Furthermore, Gpx4 downregulation in POMCₕₐ^{Δ1-26}-expressing N43/5 cells was restored by treatment with CQ and BafA1, unlike when treated with MG132 and 3-MA (FIG. 6H), confirming that POMC-induced Gpx4 degradation was associated with CMA (FIG. 6I). Actually, the expression of POMCₕₐ^{Δ1-26} significantly increased the levels of CMA components such as Lamp2a, Hsc70, and Hsp90 (FIG. 6J), and the Gpx4 protein was significantly stabilized by knockdown of a CMA receptor Lamp2a (FIG. 6K).

### 7-2. Reduction of Hspa5-Gpx4 Interaction

Since the stress-inducible heat shock factor Hspa5 (known as the major ER chaperone BiP or Grp78) was known to inhibit the degradation of Gpx4 through direct interaction with Gpx4 during ferroptosis, which was confirmed in ferroptosis due to cytosolic remaining of POMC. Specifically, N43/5 cells were transfected with 10 nM Hspa5-specific siRNA (SCBT, sc-35522) using Lipofectamine RNAiMAX (Thermo Fisher Scientific, 13778150), and after 24 hours, pCH4134 (POMCₕₐ^{Δ1-26}) (final 1 μg/ml) was transfected using Lipofectamine 2000 (Thermo Fisher Scientific, 11668019), and then the mRNA level of Gpx4 was analyzed and CHX-chase assay was performed. In addition, in order to determine whether Hspa5 counteracted POMC-induced ferroptosis in the cytosol, Hspa5_{myc}^{Δ1-18} and POMCₕₐ^{Δ1-26}, which remained in the cytosol without SP, were co-expressed in N43/5 cells and a ferroptosis induction assay was performed as in Example 6-1 above. In addition, a cytosol-organelle fractionation assay was performed as in Example 5-2 above. In addition, a co-immunoprecipitation assay was performed to determine whether POMC maintained in the cytosol promoted Gpx4 degradation by interfering with the Hspa5-Gpx4 interaction.

As the CHX-chase assay result, it was found that knockdown of Hspa5 in POMCₕₐ^{Δ1-26}-expressing N43/5 cells accelerated the degradation of Gpx4 without affecting Gpx4 mRNA (FIGS. 7A and 7B). In addition, co-expression of Hspa5_{myc}^{Δ1-18} improved lipid peroxidation and decreased cell viability caused by POMCₕₐ^{Δ1-26} (FIGS. 8B and 8C), co-expression of Hspa5_{myc}^{Δ1-18} and POMCₕₐ^{Δ1-26} restored changes in the expression levels of Gpx4, CHOP, Nox2, and Nox4 induced by POMCₕₐ^{Δ1-26} (FIG. 8D), and co-expression of Hspa5_{myc}^{Δ1-18} inhibited the degradation of Gpx4 caused by POMCₕₐ^{Δ1-26} (FIG. 8E). This meant that cytosolic Hspa5 inhibited POMC-mediated ferroptosis by preventing the degradation of Gpx4 and upregulating ER stress markers.

Furthermore, as the cytosol-organelle fractionation assay result, Hspa5 was significantly detected not only in the Triton X-100-treated organelle fractions of POMCₕₐ^{Δ1-26}-expressing N43/5 cells (FIG. 7C, Lanes 5 and 6), but also in the digitonin-treated cytosolic fractions (FIG. 7, Lanes 3 and 4). In addition, the expression of POMCₕₐ^{Δ1-26} significantly increased the level of Hspa5, a marker of ER stress response, whereas the expression of cytosolic POMCₕₐ^{Δ1-26} significantly decreased the level of Gpx4, particularly in the cytosolic fractions (FIG. 7C, Lanes 3 and 4). In addition, as the co-immunoprecipitation assay result, it was found that POMCₕₐ^{Δ1-26} bound to Hspa5_{myc} to reduce the Gpx4-Hspa5 interaction (FIGS. 7D and 7E), confirming that POMC, which was not translocated from the cytosol to ER, induced the degradation of Gpx4 by sequestering Hspa5.

### Example 8. Analysis of POMC Translocation Mechanism of Marchf6 into ER

Full-length POMCₕₐ significantly reduced the Hspa5-Gpx4 interaction in Marchf6-KO N43/5 cells (FIG. 7F, cf. Lanes 2 and 3), but this reduction was restored by Fer-1 treatment (FIG. 7F, cf. Lane 4), and thus, whether removal of Marchf6 affected ER translocation of SP-containing (ER-translocated) POMC by increasing lipid ROS levels was confirmed by differential detergent subcellular fractionation followed by IP-IB assay.

As a result, the POMCₕₐ levels in the (digitonin-extracted) cytosolic fractions of Marchf6-KO N43/5 cells were significantly reduced by Fer-1 treatment (FIG. 7G, cf. Lanes 3 and 4), whereas the POMCₕₐ levels were found to be significantly increased even in the cytosolic fractions of WT N43/5 cells when treated with erastin, a ferroptosis inducer (FIG. 7H, cf. Lanes 3 and 4).

Since erastin upregulated lipid peroxidation and induced Marchf6 inactivation, through these results, it may be estimated that Marchf6 promotes translocation of POMC into ER by preventing lipid peroxidation.

### Example 9. Analysis of Physiological Role of POMC Neuron-Specific Marchf6-Mediated POMC Degradation

To determine the physiological role of Marchf6-mediated POMC degradation in POMC neurons, POMC neuron-specific Marchf6-deficient mice (Marchf6^{POMC}) were produced using Cre-loxP technology. Specifically, POMC neuron-specific Marchf6-floxed mice were produced by crossing Marchf6^{f1/f1} mice (EUCOMM, Marchf6tm1c) without LacZ and neomycin selection cassettes with POMC-Cre mice (Jackson Laboratory, #JAX010714) under a C57BL/6J background. Genomic DNA obtained by sampling the tails of the produced mice was analyzed using PCR-based genotyping. At this time, used genotyping primer pairs (OCH5339: CACTAGACATGCTGTCAACGTGAGTATTA, OCH9075: TCAAGTAATAAGATTAAATACATGAGCCAGGC; and OCH5339: CACTAGACATGCTGTCAACGTGAGTATTA, OCH9129: GCGAGCTCAGACCATAACTTCG) were designed to detect 277-bp wild-type DNA of Marchf6 exon 5 and 248-bp floxed DNA thereof, respectively. The body weight and food intake of the produced POMC neuron-specific Marchf6-deficient mice (Marchf6^{POMC}) were measured using a digital scale each week, and body compositions such as fat mass and lean mass were measured using a quantitative NMR-based analyzer (EchoMRI, EchoMRI-700). To evaluate energy expenditures, animals were acclimated to the analysis conditions before 48 hours of analysis and then evaluated for an additional 48 hours on a 12:12 h day/night cycle using the PhenoMaster (TSE SYSTEM). The day/night cycle during this analysis was identical to that during the initial residential period of the mouse, and the same food was provided in a single metabolic cage. In addition, for the immunohistochemistry (IHC) assay, the mice were anesthetized by intraperitoneal injection of Avertin (250 mg/kg of body weight, Sigma, T48402) and transcardially perfused sequentially with PBS and 4% paraformaldehyde (PFA) (Mentos Biotechnology, M1177). The brains were removed from the mice and post-fixed in a 4% PFA solution at 4° C. for subsequent sectioning (coronal sections at 60 μm thickness) using a vibratome (Leica, VT1000S). Arcuate nucleus (ARC)-containing tissue sections were obtained according to the mouse brain atlas (https:/mouse.brain-map.org/static/atlas) and incubated with a blocking buffer solution (100 mM phosphate buffer, 4% normal donkey serum, 0.5% Triton X-100) for 30 minutes at 4° C. Thereafter, anti-Gpx4 (1:500), anti-4HNE (1:500), and anti-Hspa5 (1:500) were used as primary antibodies and treated at 4° C. for 12 hours, and the tissues were washed three times with 100 mM phosphate buffer for 10 minutes each at 4° C. and treated with Alexa-Fluor 488, 568, or 647-conjugated secondary antibody (1:500) corresponding to each primary antibody at 4° C. for 12 hours. The tissues were mounted with a mounting solution containing DAPI (Vector Laboratories, H-2000) and imaged with a laser scanning confocal microscope (Olympus, FV1000) with a size of 1024×1024 pixels, and fluorescence intensities were quantified using ImageJ (NIH). In addition, in order to measure the level of POMC-derived α-melanocyte stimulating hormone (α-MSH) in the hypothalamus of Marchf6^{POMC} mice, the hypothalamus was collected from the mice, immediately frozen in liquid nitrogen, and stored at −80° C., and the frozen hypothalamus was lysed, resuspended in 0.1 N HCl solution, and sonicated. The supernatant was obtained by centrifugation at 17,000×g at 4° C. for 20 minutes, and an ELISA assay was performed using an α-MSH ELISA kit (Phoenix Pharmaceuticals, EK-04301).

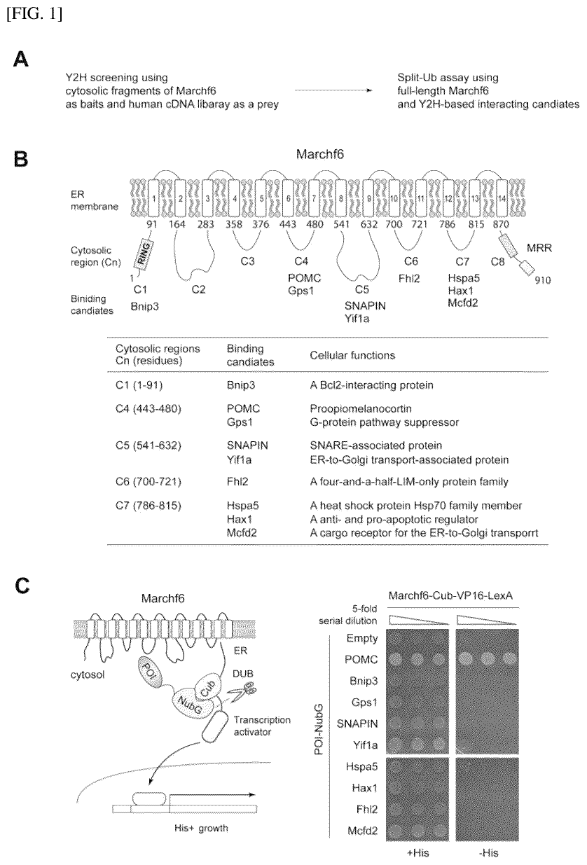

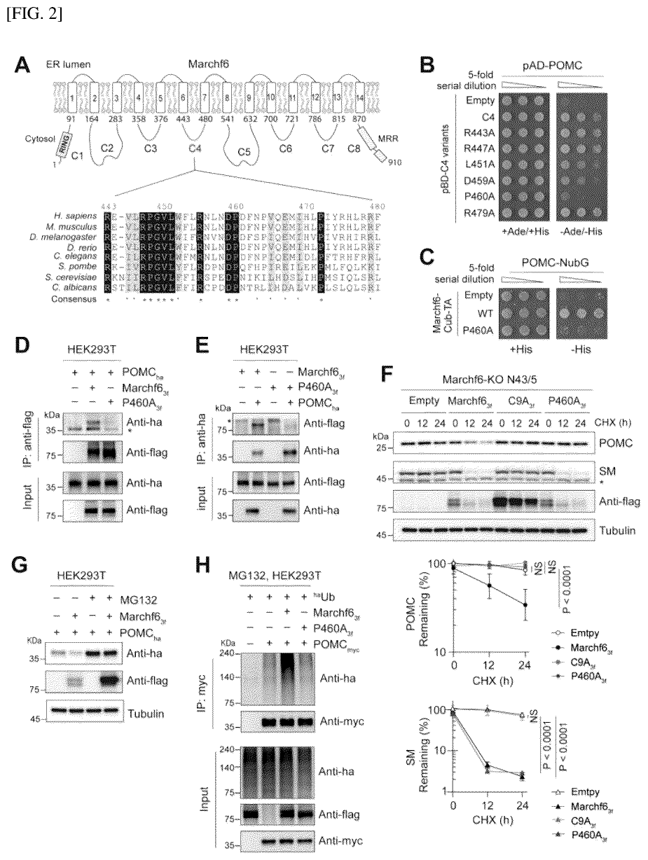

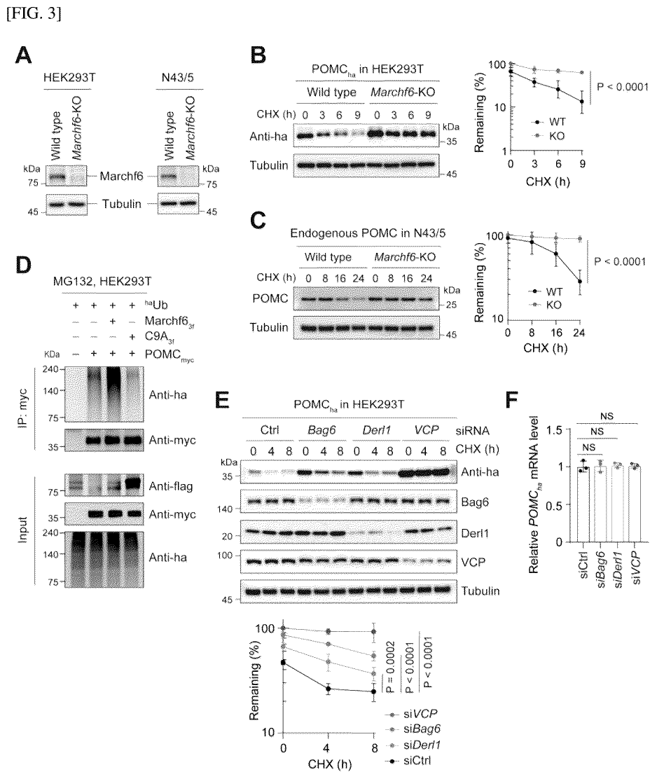

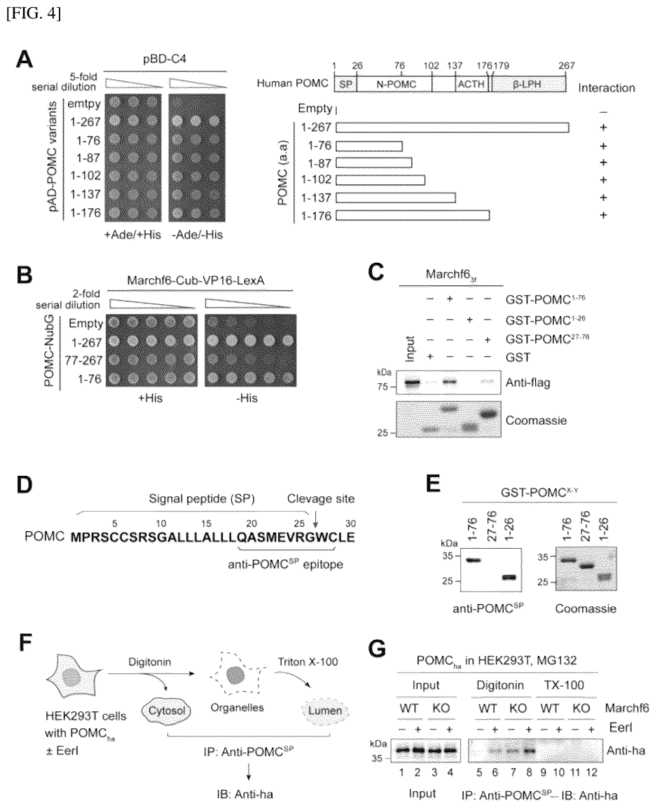

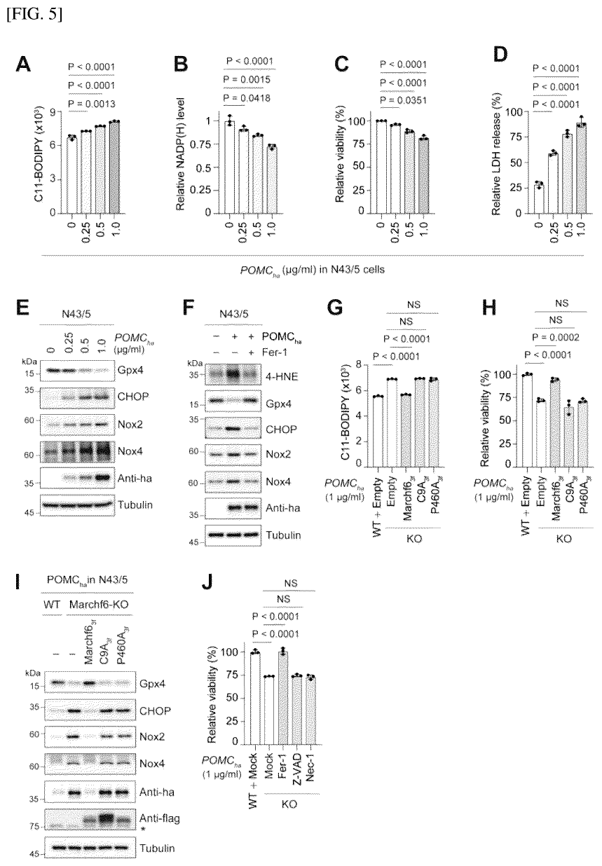

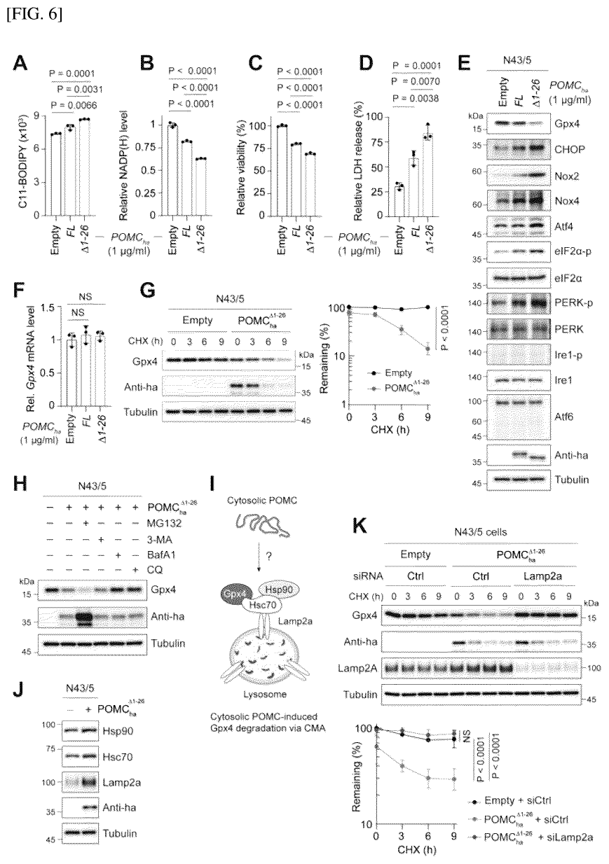

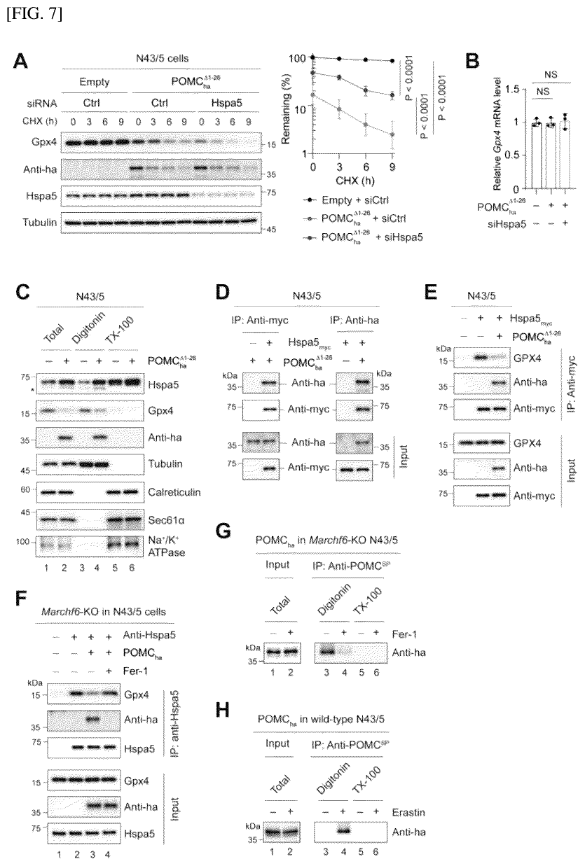

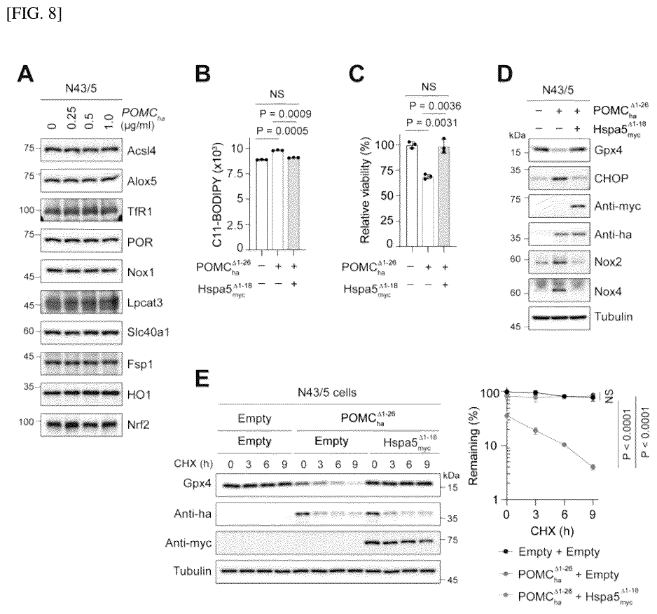

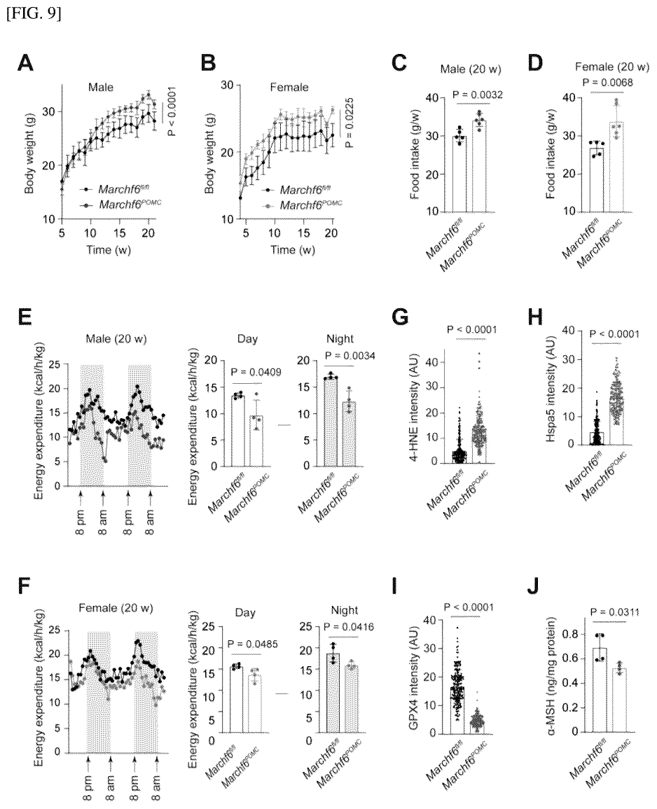

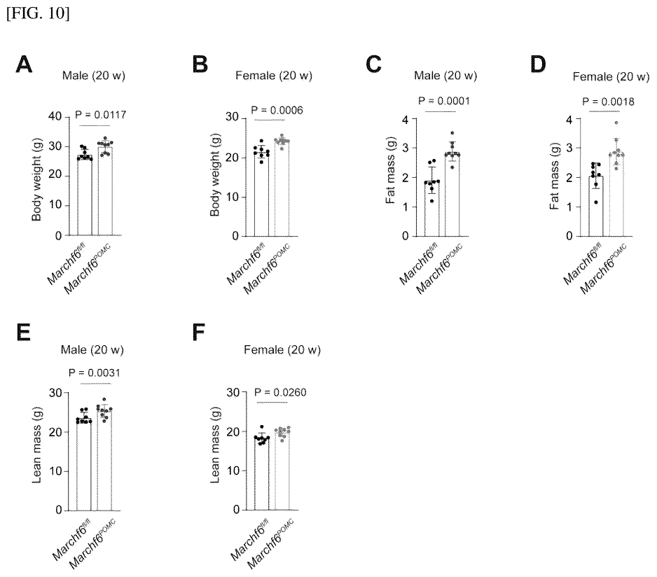

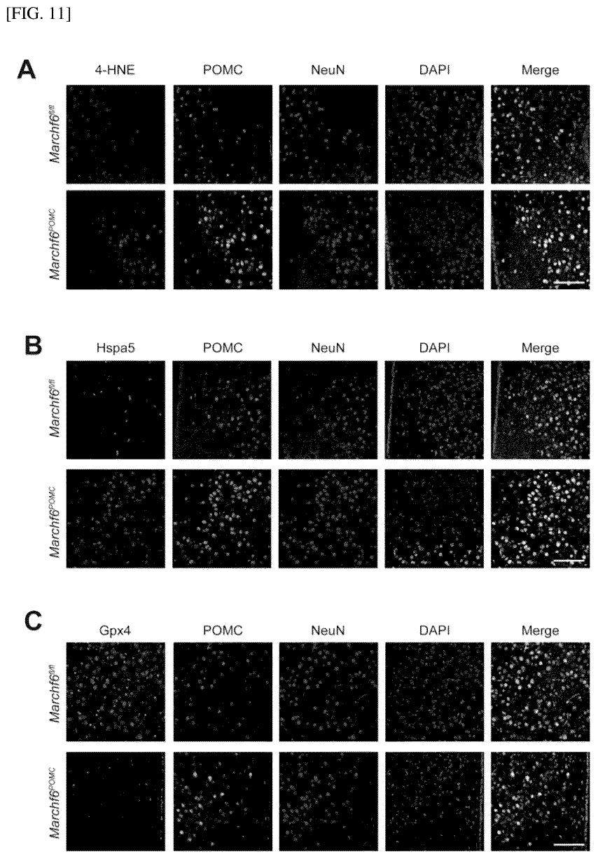

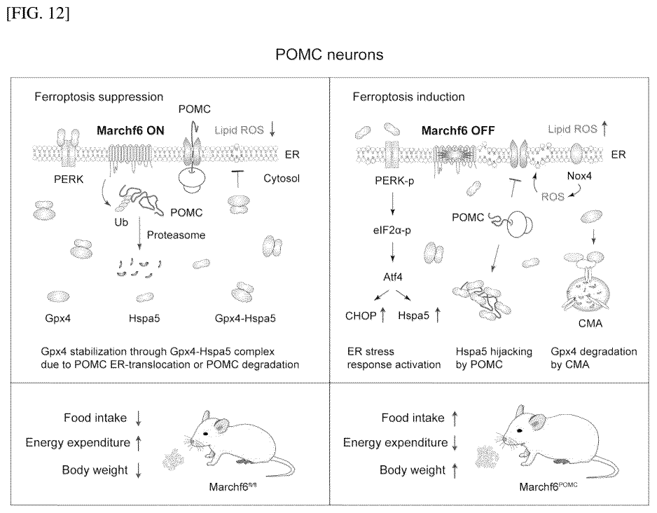

Marchf6^{POMC} mice, POMC neuron-specific Marchf6-deficient mice, were normal at birth in both male and female, but showed significantly increased body weight compared to littermate Marchf6^{f1/f1} mice under normal food intake (FIGS. 9A and 9B, and 10A and 10B). In particular, the total amount of food consumed was significantly higher in the Marchf6^{POMC} mice than in 20-week-old Marchf6^{f1/f1} mice (FIGS. 9C and 9D). In addition, as the body composition assay result, it was found that both male and female Marchf6^{POMC} mice had increased fat mass and low fat-free mass (FIGS. 10C to 10F). In contrast, energy expenditure was significantly reduced in Marchf6^{POMC} mice compared to littermate Marchf6^{f1/f1} mice (FIGS. 9E and 9F). In addition, as the IHC assay result, it was found that the levels of lipid peroxidation products 4-HNE, POMC, and Hspa5 were significantly increased in the hypothalamic ARC of Marchf6^{POMC} male mice compared to littermate Marchf6^{f1/f1} mice (FIGS. 9G and 9H, and 11), whereas the Gpx4 levels were significantly decreased in Marchf6-deficient POMC neurons (FIGS. 91 and 11). Furthermore, the POMC-derived α-MSH level in the hypothalamus of Marchf6^{POMC} mice was significantly reduced compared to Marchf6^{f1/f1} mice (FIG. 9J). Since the Marchf6^{POMC} mice had higher POMC protein levels in POMC neurons than the littermate Marchf6^{f1/f1} mice, the increased lipid peroxidation induced by Marchf6 deficiency delayed translocation of POMC into ER, resulting in reduced release of α-MSH, one of POMC-derived bioactive peptides.

Through the result, it was confirmed that POMC neuron-specific Marchf6-deficient mice exhibited hyperphagia, weight gain, and reduced energy expenditure due to increased POMC-mediated ferroptosis damage, increased ER stress, and subsequent reduction in mature POMC-derived hormones.

1. An antigenic peptide for producing a pro-opiomelanocortin (POMC)-specific antibody, comprising an amino acid sequence represented by SEQ ID NO: 1.

2. The antigenic peptide of claim 1, wherein the antigenic peptide is for producing the POMC-specific antibody including a signal peptide (SP) sequence.

3. (canceled)

4. (canceled)

5. A method for producing a POMC-specific antibody comprising:
a) inducing an immune response by inoculating a host other than a human multiple times with the antigenic peptide of claim 1; and
b) obtaining serum from blood of the host.

6. The method for producing the POMC-specific antibody of claim 5, further comprising:
purifying the POMC-specific antibody from the serum.

7. A POMC-specific antibody or antigen-binding fragment that specifically binds to POMC produced by the method of claim 5.

8. The POMC-specific antibody or antigen-binding fragment of claim 7, wherein the POMC-specific antibody or antigen-binding fragment specifically binds to POMC including an SP sequence.

9. (canceled)

10. The POMC-specific antibody or antigen-binding fragment of claim 7, wherein the POMC-specific antibody or antigen-binding fragment binds to an epitope or epitope segment including an amino acid sequence represented by SEQ ID NO: 1.

11. (canceled)

12. (canceled)

13. (canceled)

14. (canceled)

15. (canceled)

16. A pharmaceutical composition for preventing or treating diseases associated with membrane associated ring-CH-type finger 6 (Marchf6) dysfunction, comprising the POMC-specific antibody or antigen-binding fragment of claim 7 as an active ingredient.

17. The pharmaceutical composition for preventing or treating diseases associated with Marchf6 dysfunction of claim 16, wherein the diseases are diseases in which a function of Marchf6, which degrades an SP sequence of cytosolic POMC, is reduced.

18. A composition for diagnosing diseases associated with POMG accumulation, comprising the POMC-specific antibody or antigen-binding fragment of claim 7 as an active ingredient.

19. (canceled)

20. A method for providing information for diagnosing metabolic diseases, comprising detecting POMC accumulated in cytosol of POMC neurons in a biological sample isolated from a subject using the POMC-specific antibody or antigen-binding fragment of claim 7.

21. The method for providing information for diagnosing metabolic diseases of claim 20, wherein the metabolic diseases are any one selected from the group consisting of obesity, bulimia, diabetes, arteriosclerosis, hypertension, hyperlipidemia, fatty liver, metabolic liver disease, and cardiovascular disease.

22. A method for preventing or treating diseases associated with membrane associated ring-CH-type finger 6 (Marchf6) dysfunction, comprising administering the pharmaceutical composition of claim 16 to a subject in a pharmaceutically effective amount.

23. A pharmaceutical composition for preventing or treating metabolic diseases, comprising a membrane associated ring-CH-type finger 6 (Marchf6) protein or fragment thereof, or an activator or expression promoter of Marchf6 as an active ingredient.

24. The pharmaceutical composition for preventing or treating metabolic diseases of claim 23, wherein the fragment of Marchf6 includes a C4 domain.

25. The pharmaceutical composition for preventing or treating metabolic diseases of claim 23, wherein the fragment of Marchf6 includes C9 and P460 in an amino acid sequence.

26. The pharmaceutical composition for preventing or treating metabolic diseases of claim 23, wherein the expression promoter is a recombinant vector including a nucleic acid encoding Marchf6 or a fragment thereof.

27. (canceled)

28. (canceled)

29. A composition for promoting degradation of pro-opiomelanocortin (POMC), comprising a Marchf6 protein or fragment thereof, or an activator or expression promoter of Marchf6.

30. (canceled)

31. (canceled)

32. (canceled)

33. (canceled)

34. (canceled)

35. (canceled)

36. (canceled)

37. A method for preventing or treating metabolic diseases, comprising administering to a subject the pharmaceutical composition of claim 23 in a pharmaceutically effective amount.


SEQUENCE LISTING (1 sequence)

SEQ ID NO: 1 - protein, 10 aa, Homo sapiens
QASMEVRGWC